(19) United States
(12) Patent Application Publication
(10) Pub. No.: US 2022/0307005 A1
(43) Pub. Date: Sep. 29, 2022

(54) BIOFILM TRANSFORMATION

(71) Applicant: Oxford University Innovation Limited, Oxford (Oxfordshire) (GB)

(72) Inventors: Ian THOMPSON, Oxford (Oxfordshire) (GB); Wei HUANG, Oxford (Oxfordshire) (GB); Chun Kiat NG, Oxford (Oxfordshire) (GB); Ronald A. ROY, Oxford (Oxfordshire) (GB); Jason RAYMOND, Oxford (Oxfordshire) (GB)

(21) Appl. No.: 17/641,586

(22) PCT Filed: Sep. 10, 2020
(86) PCT No.: PCT/GB2020/052182

§ 371 Date: Mar. 9, 2022

(30) Foreign Application Priority Data
Sep. 10, 2019 (GB) 1913045.9

(51) Int. Cl. C12N 13/00; C12N 1/20; C12N 15/87; H01M 8/16

(52) U.S. Cl. CPC C12N 13/00; C12N 1/205; C12N 15/87; H01M 8/16

(57) ABSTRACT

The invention relates to a method for the transformation of host cells of a biofilm with heterologous nucleic acid, wherein the host cells are within the extracellular matrix of the biofilm, the method comprising: adding the heterologous nucleic acid to the biofilm; and applying inertial cavitation to the biofilm in the presence of the heterologous nucleic acid to facilitate transformation of host cells within the biofilm with the heterologous nucleic acid. The invention further relates to associated methods, uses and kits for transformation of host cells of a biofilm.

This invention relates to transforming host cells with heterologous nucleic acid within an intact biofilm, associated reagents for transformation, mechanism of transformation and industrial applications of the method.

Biofilms are a community of one or more type of microorganism, such as bacteria, fungi and protists, which can form on many different surfaces. Biofilms can form in a variety of environments including on plant and animal tissue, underwater, above ground and in the vicinity of hydrothermal vents. The common feature of suitable environments for the formation of biofilms is the presence of moisture. Whilst some biofilms are considered detrimental or harmful, such as dental plaque or biofilms which form on implanted medical devices (e.g. pacemakers), others are considered useful and may be exploited for industrial purposes (e.g. bioremediation).

Bioremediation is the use of living organisms, or their products, to treat or degrade harmful compounds. Biofilms are currently used in processes such a wastewater treatment and removal of harmful substances such as heavy metal contaminants, explosives and radioactive substances. Due to the changeable nature of industrial effluent, it is desirable to modulate the properties of biofilms so that they can be adapted to become more resilient, process particular contaminants, or be killed. Biofilms comprise of a variety of different components, such as extracellular polymeric substance (EPS) which enables the biofilm to stick together and acts as a protective barrier against UV, antimicrobials and bleach; persisters, which are bacterial cells that do not divide and are resistant to many antibiotics; and dormant cells, which are not targeted by antibiotics which require some level of cellular activity. This results in a community of cells that are difficult to modulate or fully destroy, without first breaking up the biofilm.

Biofilms are also an important feature in the body. In particular, complex and variable site-dependent bacterial ecosystems exist throughout the length of the human gastrointestinal tract, and the biofilm organisms are considered to be important in modulating the host's immune system and contributing to some chronic inflammatory diseases (Macfarlane et al. Adv Appl Microbiol. 2011; 75:111-43. doi: 10.1016/B978-0-12-387046-9.00005-0, which is incorporated herein by reference). In gastrointestinal conditions such as inflammatory bowel diseases (ulcerative colitis, Crohn's disease), it has been shown that a dysbiosis exists in microbial community structure, and that there is a reduction in putatively protective mucosal organisms such as bifidobacteria. Therefore, manipulation of mucosal communities can be beneficial in restoring normal functionality in the gut, thereby improving the immune status and general health of the host.

Transferring DNA to cells is a fundamental technique of molecular cloning, and has revolutionised molecular biology. However, in environmental microbiology, the vast majority of prokaryotes (>99%) in natural environments are unculturable, and are, therefore, not amenable to DNA delivery using traditional culture-dependent DNA delivery methods. Even when prokaryotes are able to be cultured in vitro, genetic manipulation is frequently impeded because of the lack of efficient, non-invasive, and simple methods for DNA delivery.

Biological methods for bacterial DNA transfer include conjugation, gene transformation and transduction. Physical methods for gene transfer include microinjection, particle bombardment, electroporation, laser irradiation and sonoporation by ultrasound. In practice, the most commonly used methods for bacterial gene transfer are conjugation and electroporation, along with heat shock transfer, which is mostly used with E. coli cells. Conjugation and transduction usually require a specific DNA donor or host strain to achieve bacterial DNA transfer, while gene transformation is limited to a few naturally competent groups. Electroporation is highly efficient but requires a low-ionic strength medium and a high voltage for operation. Neither method is suitable for DNA delivery into the cells in a biofilm without substantial disruption to the structure of the biofilm.

Ultrasound DNA delivery (UDD) as an approach for plasmid or DNA fragment delivery has been intensively studied in recent years. While the exact mechanism of UDD remains elusive, it is possible that high frequency vibration from the ultrasound could create non-uniform stretching and compression of the cells, which physically generates reversible and transient porosity in the cell membrane. It is more likely that ultrasound induced gas and vapor cavity formation leads to spatially localized, high energy phenomena such as shock waves than can stress cell membranes, micro-streaming that can disrupt boundary layers, and micro-jetting that can puncture cell membranes. One of the attractions of UDD is that it can, in theory, deliver DNA or RNA to any type of cell including bacteria, fungi, plants and mammalian cells. However, the major disadvantage of UDD is that greater efficiency at transforming target, or host, cells is concomitant with lowered survival rates and with disruption of both the transforming DNA and DNA in the host. For example, see the Han W. Y. et al (Applied and Environmental Microbiology, June 2007, p. 3677-3683, Vol. 73, No. 11) report on an ultrasonication technique involving 1 MHz sonication and finding that the target Fuscobacterium nucleatum suffered double cross-over allelic exchanges, and the rate of transformation was 0.05 per μg DNA. These levels are extremely low, and required the use of a contrast agent even then.

The process for the transformation of planktonic bacterial cells, theoretically extrapolated to include those derived from biofilms, with heterologous nucleic acid has been described in patent GB 2452543. The process requires extraction and washing of the biofilm cells prior to ultrasound assisted transformation with heterologous nucleic acid. Whilst the technique taught by GB 2452543 is useful for transforming cells extracted from the biofilm, its application is limited. This is because the biofilm is broken up prior to transformation of the cells, which means that it necessitates the destruction of an established biofilm. Such transformed cells are also difficult to seed back into an established biofilm. The transformed biofilm-derived cells may be used to establish a new biofilm, but this is time consuming and inefficient.

What is required is an improved method of transforming cells of a biofilm such that biofilms can be genetically manipulated. Therefore, the aim of the invention is to provide an improved method of transformation of cells of a biofilm.

### SUMMARY OF INVENTION

According to a first aspect of the present invention, there is provided a method for the transformation of host cells of a biofilm with heterologous nucleic acid, wherein the host cells are within the extracellular matrix of the biofilm, the method comprising:
the addition of heterologous nucleic acid to the biofilm; and
applying inertial cavitation to the biofilm in the presence of the heterologous nucleic acid to facilitate transformation of host cells within the biofilm with the heterologous nucleic acid.

The invention advantageously provides the ability to transform cells in a biofilm in situ, which does not result in the destruction or reconstitution of the biofilm. The invention makes use of inertial cavitation, which is a phenomenon in which rapid changes of pressure in a liquid lead to the formation of small vapour-filled cavities, in places where the pressure is relatively low. When subjected to higher pressure, these cavities, called “bubbles” or “voids”, collapse and can generate an intense shock wave. Without being bound by theory, this cavitation shockwave has the ability to disrupt the biofilm and cell membranes in the biofilm sufficiently to facilitate transformation, but importantly it can avoid disintegration of the biofilm itself. In addition to avoiding the destruction of the biofilm to transform cells, the biofilms itself can be transformed in situ. The ability to transform biofilms in situ opens up the possibility to adapt biofilm properties with little or no down-time in industrial processes. Previous methods relied on the destruction of the biofilm and required extraction of cells, transformation, and then reconstitution of a biofilm, which is time consuming and leads to longer downtime where a functional biofilm is not present.

Biofilm Location

In one embodiment, the biofilm is in situ. For example, the biofilm may be in its natural environment. The term in situ is understood to mean that the biofilm is not extracted/removed from the position where it established.

The biofilm may be situated in any location where it may be temporarily enclosed or at least partially enclosed such that the heterologous nucleic acid may be incubated, without being washed away, and subsequently exposed to ultrasound.

The biofilm may be a biofilm at any location where a biofilm can benefit a natural or industrial process, such as a decontamination process. In another embodiment, the biofilm may be undesirable, for example in cases of biofouling, where it is desirable to control, reduce or eliminate the biofilm.

The biofilm may be in a watercourse, such as in a pipe. The watercourse may be open, such as a channel, or closed such as in a pipe. The watercourse may be natural or manmade. The biofilm may be on a submerged surface or in the air-liquid interface (i.e. pellicle). The biofilm may be in oil or water feed. In one embodiment, the biofilm is in a natural environment, such as a stream, river, or a water body, such as a pond, lake, reservoir or sea.

In one embodiment, the biofilm is in a waste-water treatment stream, for example flowing a feed-water there through. In another embodiment, the biofilm may be in an oil or gasoline stream, such as an oil or gasoline pipe.

In another embodiment, the biofilm may at a site of contamination, such as an oil or gasoline contaminated site. The biofilm may be in a biofilm supported leaching process, such as heap leaching, for example the biofilm comprising bacteria that oxidizes ore, releasing water soluble metals, such as cupric ion (copper).

In another embodiment, the biofilm may be in a reactor, for example in a domestic or industrial wastewater treatment plant. For example, the biofilm may be in a dispersed/suspended growth system, such as an activated sludge system or extended aeration system, or the biofilm may be in an attached growth system, such as a trickling filter, rotating biological contactor (RBC) system, or membrane bioreactor.

In another embodiment, the biofilm may be in any of the group comprising a contaminated aquifer, a pipe, the body of a ship or boat, soil crumbs, a plant leaf surface and plant roots.

In one embodiment, the biofilm is in a microbial fuel cell (MFC). The skilled person will recognise that a microbial fuel cell is a bio-electrochemical system that drives an electric current by using bacteria and a high-energy oxidant such as O□, for example mimicking bacterial interactions found in nature. In MFCs, the electrons released by bacteria from the substrate oxidation in the anode compartment (the negative terminal) are transferred to the cathode compartment (the positive terminal) through a conductive material. In the cathode, the electrons are combined with oxygen and the protons diffused through a proton exchange membrane.

The invention recognises that biofilms exist in vivo, such as throughout the gastro-intestinal tract of the body. Therefore, in one embodiment, the biofilm may be in vivo, for example in the gastrointestinal tract of a subject. The method of the invention may be carried out in vivo. The biofilm may be within the gastrointestinal tract of a mammalian subject, such as a human or non-human animal. A non-human animal may be livestock or domestic animals.

Biofilm Properties

The biofilm may be intact (e.g. it has not been previously disrupted before applying inertial cavitation to the biofilm). The biofilm may comprise or consist of bacterial cells or a combination of bacterial and fungal and/or protist cells. The host cell may be a bacterial cell, for example a bacterial cell of the biofilm. In another embodiment, the host cell may be fungal or protest.

The bacteria of the biofilm may comprise any bacteria that are capable of forming a biofilm. The bacteria of the biofilm may comprise any bacteria that produces and can reside within an extracellular matrix of extracellular polymeric substances. The bacteria may be gram positive or gram negative, or a mixture thereof. The bacteria may be cyanobacteria. Gram-positive bacteria may comprise any of the group of bacteria selected from Bacillus spp, Listeria monocytogenes, Staphylococcus spp, and lactic acid bacteria, such as Lactobacillus plantarum and Lactococcus lactis, or combinations thereof. Gram-negative bacteria may comprise Escherichia coli and/or Pseudomonas spp., such as P. aeruginosa). The Pseudomonas spp., may be P. putida, such as P. putida UWC1.

The bacteria of the biofilm may comprise Shewanella spp. such as Shewanella oneidensis. In one embodiment, the bacteria of the biofilm comprises Shewanella oneidensis MR-1.

The skilled person will recognise that the successful transformation of bacteria in a biofilm is dependent on the plasmid selected, and would be expected to use the appropriate plasmid. The skilled person will further understand that while many different types of bacteria may take up the selected plasmid, only those that can use the plasmid will elicit phenotypical or functional changes.

In one embodiment, the bacterial species to be transformed may be any bacterial species selected from the group comprising Bacillus spp, Clostridium spp, Thermus spp, Pseudomonas spp, Acetobacter spp, Micrococcus spp, Leuconostoc spp, Shewanella spp, Escherichia spp, Acidithiobacillus spp, or combinations thereof. The bacteria to be transformed may be selected from Proteobacteria, gram negative bacteria and gram-positive bacteria, or combinations thereof. The Pseudomonas spp., may be P. putida, such as P. putida UWC1.

In an embodiment wherein the bacterial species to be transformed are gut bacteria, the bacteria to be transformed may be any bacteria selected from the group comprising Proteobacteria, gram negative bacteria and gram-positive bacteria, or combinations thereof. In an embodiment wherein the bacteria to be transformed are gut bacteria, the bacteria to be transformed may be pathogenic bacteria. Alternatively, in an alternative embodiment wherein the bacteria to be transformed are gut bacteria, the bacteria to be transformed may be Firmicutes, Bacteroidetes, Actinobacteria, Proteobacteria. In an embodiment wherein the bacteria to be transformed are gut bacteria, the bacteria to be transformed may be of the genus selected from Bacteroides, Clostridium, Faecalibacterium Eubacterium, Ruminococcus, Peptococcus, Peptostreptococcus, Bifidobacterium, Escherichia, and Lactobacillus.

The content of the biofilm may be unknown, or partially unknown. For example, the bacterial species present in the biofilm may not be known prior to transformation. The heterologous nucleic acid may be suitable for transformation and use in one or more different bacterial species, such as a plurality of bacterial species. In one embodiment, the heterologous nucleic acid may be universally suitable for transformation and use in all known bacterial species. Suitable for transformation is understood to mean that the nucleic acid is functional in the host cell to be transformed, such that it has the desired effect. For example, the nucleic acid may be methylated to protect it from degradation once internalised in the cell. In another embodiment any promoter, encoded gene, encoded polypeptide, or regulatory element may be functional in the cell.

In an embodiment wherein the biofilm comprises a mixture of organisms or species, the different organisms or species may be in the biofilm as separate microcolonies, as a coaggregation, or they may be layered.

In one embodiment, the bacteria of the biofilm are capable of redox (prior to and/or after transformation according to the invention) to generate electricity in a microbial fuel cell (MFC). In one embodiment, the bacteria of the biofilm have an extracellular electron transport pathway (prior to and/or after transformation according to the invention) to generate electricity in a microbial fuel cell (MFC).

Inertial Cavitation

The inertial cavitation may be acoustically induced. In one embodiment, the inertial cavitation comprises inertial acoustic cavitation. The skilled person will readily be able to provide inertial cavitation for a given biofilm situation. For example, the inertial acoustic cavitation can be induced with ultrasound, whereby the ultrasound applied at an appropriate frequency will bring the liquid in the area of the biofilm to a cavitation threshold. The skilled person will also recognise that the parameters of the ultrasound necessary to reach the cavitation threshold in a biofilm can vary depending on the exact nature of the surrounding liquid, the presence of particles for nucleation, and the shape and size of the biofilm.

In one embodiment, the level of cavitation activity is monitored, for example by sensing the acoustic cavitation noise, and that information may be used to adjust exposure parameters in real time.

In an embodiment using ultrasound to induce inertial cavitation, the ultrasound may be applied from an ultrasound generator. The ultrasound generator may comprise an instrument that produces ultrasound frequency between 1 kHz to 2 MHz, or beyond. One or more ultrasound generator may be held at positions sufficiently near to the biofilm to effectively apply the inertial cavitation to the biofilm.

In an embodiment wherein the biofilm is in vivo, the ultrasound generator may be provided in a capsule to be swallowed and traverse the gastrointestinal tract. In another embodiment, an endoscopic ultrasound probe may be provided to induce inertial cavitation in the biofilm.

The inertial cavitation induced by ultrasound may be applied to an area or a volume of biofilm depending on the spatial distribution of the sound field, which in turn depends on the acoustic frequency, the pressure amplitude, and the geometry of the exposure vessel and ultrasound generator. The range of biofilm area and volume that can be subjected to inertial cavitation can be tailored to suit the needs of the users, by varying the frequency and the ultrasound generator geometry, and by using multiple generators to increase the range and coverage.

The aim may be to expose all the biofilm to inertial cavitation and plasmid in order to increase the percentage of bacteria that are transformed. Transformation efficiency is around 10⁻⁵%, but due to selection pressure, transformed cells will outgrow non-transformed cells to eventually change the properties of the biofilm. The skilled person will recognise that in many cases, only a small percentage of resistance cells (<1%) is needed in a biofilm to confer a resistance trait in the entire biofilm. Hence, transformation of small percentage of cells within the biofilm can induce changes in the phenotypic and functionality of the entire biofilm.

The method may comprise an incubation period between adding the heterologous nucleic acid and applying the ultrasound. The incubation period may be at least 30 seconds. In another embodiment, the incubation period may be at least 1 minute. In another embodiment, the incubation period may be at least 3 minutes. In another embodiment, the incubation period may be at least 5 minutes. In another embodiment, the incubation period may be at least 10 minutes. In another embodiment, the incubation period may be about 15 minutes, or more. In another embodiment, the incubation period may be between about 5 seconds and 30 minutes. In another embodiment, the incubation period may be between about 5 seconds and 20 minutes.

Enclosure

In one embodiment, the biofilm, or part thereof, is enclosed by an enclosure and the heterologous nucleic acid is added into the enclosure for the transformation. In one embodiment, an enclosure is applied to the biofilm when adding the heterologous nucleic acid to the biofilm. The enclosure may be held in position on the biofilm during an incubation period after the heterologous nucleic acid has been added. The incubation period within an enclosure may be at least 30 seconds. In another embodiment, the incubation period within an enclosure may be at least 1 minute. In another embodiment, the incubation period within an enclosure may be at least 3 minutes. In another embodiment, the incubation period within an enclosure may be at least 5 minutes. In another embodiment, the incubation period within an enclosure may be at least 10 minutes. In another embodiment, the incubation period within an enclosure may be about 15 minutes, or more. The ultrasound may be applied during or after the incubation period within an enclosure. The ultrasound may be applied prior to removal of the enclosure.

Enclosing the biofilm, or part thereof, advantageously contains the heterologous nucleic acid to prevent it dispersion away from the biofilm. This can be particularly beneficial in a flowing system, such as within a flowing pipe, in order to avoid heterologous nucleic acid being swept away. Providing an enclosure can be more advantageous if biofilm volume is less than ˜5% of the total liquid volume surrounding the biofilm, to reduce the amount of plasmids used and lower the operation cost. In embodiments where biofilm volume percentage is more than 5% of total liquid volume, or where cost of the operation is not a primary concern for its adoption, such as for medical application to be used in a living gut, an additional enclosure, beyond natural gut walls, may not be used.

The enclosure may be used if the biofilm volume is less than about 5% of the total liquid volume surrounding the biofilm.

The enclosure may comprise a water-impermeable or partially permeable barrier, such as glass, ceramic, plastic or rubber. The enclosure may block the passage of nucleic acid, such as plasmids, but may be permeable to water. Materials such as a membrane which blocks plasmids but permeable to water can be used. The enclosure may be an open ended container, such as a cup or dome that can be held over the biofilm. For example, the enclosure may be a container that can be held against a surface in situ to enclose the biofilm, or part thereof, and prevent the heterologous nucleic acid from dispersing away. In one embodiment, the enclosure is flexible, at least in part. The flexible part of the enclosure may be a part that is arranged to contact the biofilm surface, or the surface that the biofilm is on. For example the rim of the enclosure may be flexible or otherwise shape-conforming for example when held against a surface. The flexibility of the enclosure may be to enable it to conform to a surface, for example, in situ.

The enclosure may comprise an access port for administration/delivery of the heterologous nucleic acid, and/or other substances, into the enclosure. Additionally or alternatively, the enclosure may comprise a secondary enclosure that is arranged to retain and release the heterologous nucleic acid and/or other substances into the enclosure.

The skilled person will recognise that the size of the enclosure is dependent on the size of the biofilm or the area of biofilm to be transformed. It can be used to segregate biofilm from the bulk of the surrounding liquid content. Therefore, in one embodiment, the size of the enclosure is from about 0.5 cm³ to about 250 cm³. In another embodiment, the size of the enclosure is from about 0.5 cm³ to about 500 cm³. In another embodiment, the size of the enclosure is from about 0.5 cm³ to about 300 cm³. In another embodiment, the size of the enclosure is from about 250 cm³ to about 350 cm³. In another embodiment, the size of the enclosure is about 300 cm³.

In one embodiment, the enclosure is attached to the ultrasound generator. The ultrasound generator may be surrounded by the enclosure, such that it protrudes into the enclosure. In another embodiment, the enclosure is attached to one or more ultrasound generators. The skilled person will recognise that the number of generators required is dependent on the effective coverage/range of the ultrasound generator and the size of the enclosure.

Heterologous Nucleic Acid

The heterologous nucleic acid may be DNA or RNA. Additionally or alternatively, the heterologous nucleic acid may comprise nucleotide analogues, such as locked nucleic acid (LNA) or bridged nucleic acid (BNA), morpholino, and peptide nucleic acid (PNA).

In one embodiment, the heterologous nucleic acid is a plasmid or vector. The plasmid may be a bacterial plasmid. In another embodiment, the heterologous nucleic acid may be linear.

The heterologous nucleic acid may be provided at a concentration of at least about 0.1 to about 10 ng/μL of enclosed volume.

The heterologous nucleic acid may encode a gene and/or or a regulatory element that is capable of modifying the host cell phenotype (i.e. capable of genetic modification). The heterologous nucleic acid may be arranged to knockout a host cell gene, or a regulatory sequence thereof, of the host cell. The heterologous nucleic acid may encode more than one gene and/or regulatory element. The heterologous nucleic acid may be bacterial in origin or sequence, or may be arranged to express a polypeptide in a bacterial cell. The heterologous nucleic acid may encode a bacterial gene and/or or a bacterial regulatory element that is capable of modifying a bacterial host cell phenotype (i.e. capable of genetic modification).

The heterologous nucleic acid may encode a gene and/or regulatory element involved in, or that is arranged to modify, quorum sensing, cell metabolism, heat/cold resistance, heat-shock resistance, chemical resistance, antibiotic resistance, cell aggregation, cell adhesion, cell export, membrane transport molecules, cell or EPS dispersal enzymes, and stress regulons; or combinations thereof.

The heterologous nucleic acid may encode a gene and/or regulatory element involved in, or that is arranged to modify, the host cells metabolism, production or export of molecules selected from surfactant, lipid, polysacharride, protein, and DNA. The heterologous nucleic acid may encode a gene and/or regulatory element of a gene that can facilitate the metabolism of and/or resistance to contaminants. The heterologous nucleic acid may encode a gene and/or regulatory element of a gene that can facilitate the metabolism of and/or resistance to petroleum hydrocarbons and/or chlorinated organics.

The heterologous nucleic acid may encode an enzyme, a membrane transporter, a pore molecule, and/or a regulatory element associated therewith.

The genetic modification may comprise augmentation of a gene or phenotypic property. In particular, said gene or phenotypic property of the host cell may be enhanced, for example by increased or decreased expression of the gene.

The heterologous nucleic acid may encode a gene and/or regulatory element of a gene selected from any of the group comprising protein-degrading enzymes, such as protease and peptidase; polysaccharide-degrading enzymes and oligosaccharide-degrading enzymes, such as endocellulase, chitinase, α-glucosidase, β-glucosidase, β-xylosidase, N-acetyl-β-d-glucosaminidase, chitobiosidase, and β-glucuronidase; lipid-degrading enzymes, such as lipase and esterase; phosphomonoesterases, such as phosphatase; oxidoreductases, such as phenol oxidase, peroxidase; and extracellular redox activity; or combinations thereof.

The gene and/or regulatory element of a gene may encode one or more functions selected from the group comprising radioactive compound degradation, metal oxidation or reduction, aromatic compound degradation, surfactant production or degradation, nuclease resistance, antimicrobial resistance, metal resistance, aromatic compound resistance, surfactant resistance, changes in stickiness of biofilm, promotion/prevention of biofilm dispersal, production of light/fluorescence emitting matter, production of organic matter, production of organometallic matter, production of polymeric substances, and production of energy (such as biofuels, biogas, bioelectricity etc.). The skilled person will recognise that these features do not comprise an exhaustive list of functions of a transformed biofilm and that the function encoded by the gene and/or regulatory element may be selected dependent on the intended use of the biofilm.

The gene may encode an enzyme. The gene may encode an enzyme selected from the group comprising P450, Cytochrome, Oxidoreductase, Transferase, Hydrolase, Lyase, Isomerase, and Ligase.

The heterologous nucleic acid may encode one or more, or all, components of a redox pathway, such as an extracellular electron transport pathway. The heterologous nucleic acid may encode one or more, or all, components of a flavin synthesis pathway. For example, the heterologous nucleic acid may encode the gene cluster ribADEHC, or one or more individual genes thereof. The gene cluster ribADEHC may be cloned from any suitable bacteria, such as Bacillus subtilis. In another embodiment, the heterologous nucleic acid may encode one or more, or all, components of the mtrCAB pathway (an extracellular electron transport pathway).

The heterologous nucleic acid may be arranged to promote survival or growth of a selected species of bacteria in the biofilm relative to other species. For example where a particular species is beneficial for an industrial process, a species that facilitated the process may be promoted to survive or grow in the biofilm.

The heterologous nucleic acid may further encode a selectable marker and/or reporter gene. The selectable marker may be an antibiotic resistance gene, such as resistance to kanamycin. The reporter gene may comprise a fluorescence marker, such as GFP or luciferase. The selectable marker and/or reporter gene may be used to determine if transformation is successful and/or to maintain a selection for modified cells in the biofilm). The methods or uses herein may comprise the use of selective pressure to select for successful transformants in the biofilm. The heterologous nucleic acid may be arranged to insert into or otherwise knockout a gene or regulatory sequence that can be used as a marker for transformation. The skilled person will recognise that the choice of selectable marker or reporter gene is dependent on the intended application of the transformed biofilm.

In one embodiment, the heterologous nucleic acid may comprise homologous sequence, which is homologous to the host cell nucleic acid. For example, the homologous sequence may be provided for homologous recombination in order to integrate the heterologous nucleic acid into the host cell nucleic acid, such as the chromosome. Such heterologous recombination may be to insert a functional gene and/or create a knockout mutant of one or more genes of the host cell.

The heterologous nucleic acid may be of any suitable size for transformation. In one embodiment, the heterologous nucleic acid is between 500 bp and 20 kbp. In another embodiment, the heterologous nucleic acid is between 1 kbp and 15 kbp. In another embodiment, the heterologous nucleic acid is less than 40 kbp, 30 kbp or 20 kbp.

Other Method Variables

The heterologous nucleic acid may be applied to the biofilm in a CaCl₂ solution. The CaCl₂ solution may be provided in a concentration of about 50 mM. In another embodiment, the CaCl₂ solution may be provided in a concentration of between about 10 mM and about 200 mM. In another embodiment, the CaCl₂ solution may be provided in a concentration of between about 10 mM and about 150 mM. In another embodiment, the CaCl₂ solution may be provided in a concentration of between about 10 mM and about 100 mM. In another embodiment, the CaCl₂ solution may be provided in a concentration of between about 20 mM and about 80 mM. In another embodiment, the CaCl₂ solution may be provided in a concentration of between about 30 mM and about 70 mM. In another embodiment, the CaCl₂ solution may be provided in a concentration of between about 40 mM and about 60 mM.

Once the transformation is carried out, the enclosure may be removed from the biofilm.

Biofilm Properties After Transformation

Transformed biofilm may comprise a mixture of transformed and non-transformed cells. Transformation of a biofilm is considered to be successful when the biofilm has at least a 10% improvement in the modified functionality compared to non-transformed biofilm. E.g. ≥10% reduction in loss of biomass after introduction/increase in concentration of contaminant in the feed solution. In another example, ≥10% increase in degradation of contaminant. In one embodiment, not all cells of the biofilm need to be transformed. For example less than 5% of the cells of a biofilm may be transformed with antibiotic resistance to ensure that the biofilm as a whole is resistant.

The success of transformation of cells in the biofilm may be determined by its beneficial effect. For example, in an industrial wastewater treatment plant, when introduced with water containing higher than usual aromatic compound, a successful transformed biofilm may be considered to be able to survive and not detached more than 30% of its biomass, as compared to its non-transformed counterpart.

Other Aspects

Method of Adapting a Biofilm

According to another aspect of the present invention, there is provided a method of adapting a biofilm in situ, the method comprising transformation of host cells within the extracellular matrix of the biofilm with heterologous nucleic acid,
wherein the method comprises:
adding the heterologous nucleic acid to the biofilm, optionally within an enclosure applied to the biofilm; and
applying inertial cavitation to the biofilm in the presence of the heterologous nucleic acid to facilitate transformation of host cells within the biofilm with the heterologous nucleic acid,
wherein the heterologous nucleic acid encodes a gene and/or or a regulatory element that is capable of modifying the host cell phenotype, or the heterologous nucleic acid is arranged to knockout a host cell gene, or regulatory sequence thereof, of the host cell.

In an embodiment wherein the method is carried out in vivo, for example in a mammalian subject, the method may be therapeutic or non-therapeutic.

Method of Decontamination of Waste Water Feedstock

According to another aspect of the present invention, there is provided a method of decontaminating feedstock in a waste water treatment process, the method comprising:
flowing the feedstock over a biofilm,
wherein cells of the biofilm have been genetically modified in situ in order to increase their ability to reduce the contaminant, such as an aromatic, in the feedstock and/or increase the resistance of the biofilm to the contaminant in the feedstock.

The method may comprise the step of modifying cells of the biofilm according to the invention herein. Modifying cells of the biofilm may be provided by the method of the invention herein. For example, the biofilm, or a portion thereof, may be enclosed and plasmids for transformation may be added to the enclosed biofilm. The method may further comprise incubating the enclosed biofilm and plasmids, for example for about 15 minutes, and exposing the enclosed biofilm and plasmids to ultrasound. The method may further comprise removing the enclosure from the biofilm and allowing the contaminated feedstock to interact with the biofilm.

The method may comprise the step of detecting/monitoring the contaminant in the feedstock before and/or after transformation of the cells of the biofilm.

The plasmids for transformation may encode one or more genes or gene regulators that can increase resistance to the contaminant and/or increase the biofilm's ability to degrade the contaminant. Reducing the contaminant in the feedstock may comprise degrading the contaminant, sequestering the contaminant, absorbing the contaminant, or converting the contaminant. For example, the cells of the biofilm may be transformed with nucleic acid encoding a heterologous polypeptide, or nucleic acid encoding a regulatory element for upregulating an endogenous polypeptide, wherein such polypeptide may have the ability to degrade, sequester, absorb, or convert the contaminant.

According to another aspect of the present invention, there is provided a method of modifying the bacteria of a microbial fuel cell (MFC), wherein the bacteria are in a biofilm, the method comprising transforming the bacteria of the biofilm with heterologous nucleic acid encoding one or more genes of a redox pathway.

The redox pathway may be a Flavin biosynthesis pathway. For example, the heterologous nucleic acid may encode the gene cluster ribADEHC, or one or more individual genes thereof. The gene cluster ribADEHC may be cloned from any suitable bacteria, such as Bacillus subtilis.

According to another aspect of the present invention, there is provided a method of generating electricity from a microbial fuel cell (MFC) comprising:
culturing bacteria in a biofilm in an anode compartment of the MFC, wherein the bacteria of the biofilm have been transformed with heterologous nucleic acid encoding one or more genes of a redox pathway;
supplying an oxidant and a substrate for oxidation that are substrates of the redox pathway;
generating electricity by allowing electrons released by the bacteria from the substrate oxidation in the anode compartment to be transferred to a cathode compartment of the MFC through a conductive material, whereby the transferred electrons in the cathode compartment are combined with oxygen and the protons are diffused through a proton exchange membrane.

The redox pathway may be an extracellular electron transport pathway.

Use

According to another aspect of the present invention, there is provided the use of inertial cavitation to a biofilm in the presence of heterologous nucleic acid to transform host cells within the extracellular matrix of the biofilm.

The use may be to transform cells of a biofilm in situ. The use may be to transform cells of a biofilm in a microbial fuel cell (MFC) for generation of electricity.

Kit

According to another aspect of the present invention, there is provided a kit for transformation of host cells within the extracellular matrix of a biofilm, wherein the kit comprises:
an inertial cavitation generator;
an enclosure; and
optionally, nucleic acid for transformation.

According to another aspect of the present invention, there is provided a kit for transformation of host cells within the extracellular matrix of a biofilm, wherein the kit comprises:
an inertial cavitation generator;
nucleic acid for transformation; and
optionally an enclosure.

The inertial cavitation generator may comprise an ultrasound generator. The nucleic acid for transformation may be bacterial in sequence, or arranged to express a bacterial gene and/or bacterial regulatory element.

The kit may further comprise CaCl₂.

The kit may further comprise a test kit for determining successful transformation comprising a sample bottle and/or a UV light (e.g. if a fluorescent marker used, such as GFP).

According to another aspect of the invention, there is provided a microbial fuel cell (MFC), wherein the microbial fuel cell comprises a biofilm that has been modified by transformation with heterologous nucleic acid into the bacteria of the biofilm according to the method herein.

The bacteria of the biofilm in the microbial fuel cell (MFC) may be transformed with nucleic acid encoding one or more, or all, components of a redox pathway, such as an extracellular electron transport pathway. The redox pathway may comprise a Flavin biosynthesis pathway. For example, the heterologous nucleic acid may encode the gene cluster ribADEHC, or one or more individual genes thereof. The gene cluster ribADEHC may be cloned from any suitable bacteria, such as Bacillus subtilis. In another embodiment, the bacteria of the biofilm in the microbial fuel cell (MFC) may be transformed with nucleic acid encoding one or more, or all, components of the mtrCAB pathway (an extracellular electron transport pathway).

Definitions

The term “biofilm” describes any syntrophic community of microorganisms in which the constituent cells are stuck to each other and often also to interfaces, such as air-liquid, air-solid and liquid-solid interface. The constituent cells become embedded within a slimy extracellular matrix that is composed of extracellular polymeric substances, produced by the constituent cells. The biofilm may include a single species or a diverse group of microorganisms. Microorganisms of the biofilm may include, but are not restricted to, bacteria, archaea, protozoa, fungi and algae.

The skilled person will understand that the extracellular matrix of the biofilm comprises extracellular polymeric substances (EPS). EPS components are typically a polymeric conglomeration of extracellular polysaccharides, proteins, lipids and DNA.

As used herein, the term “heterologous nucleic acid” relates to a length of nucleic acid that it is desired to incorporate into the host cell. The nucleic acid may be either DNA or RNA, and may range from an oligomer up to a plasmid, for example. Indeed, the present invention is particularly suitable to incorporate plasmids into host cells. In particular, heterologous nucleic acid is any nucleic acid that it is desired to introduce into the host cell, and will not generally already be present in the host cell or, if it is, then it is present in a form different from that being introduced.

The term “transformation” relates to the introduction of the nucleic acid sequence into the host cell, and the transformed host may have the nucleic acid present as a free plasmid, for example, or the nucleic acid may be incorporated into the host genome. In any event, it is preferred that the nucleic acid be of the same type as the host genome, unless otherwise required. Thus, it is generally preferred to transform mammalian cells with DNA, for example. However, it is also preferred that the nucleic acids can include linear or circular DNA or RNA.

The skilled person will recognise that ultrasound is sound waves with frequencies higher than the upper audible limit of human hearing. This limit varies from person to person and is approximately 20 kilohertz (20,000 hertz) in healthy young adults. Ultrasound generators can operate with frequencies from 20 kHz up to several gigahertz.

The skilled person will recognise that the application of sufficiently intense ultrasound to a liquid medium can result in the formation of highly energetic gas and vapor cavities, a process known as acoustic cavitation. These cavities can concentrate energy spatially, generate acoustic shock waves, induce microjet formation, and induce microstreaming that can disrupt boundary layers. All of these effects promote transfection and can also induce cell death.

The skilled person will understand that optional features of one embodiment or aspect of the invention may be applicable, where appropriate, to other embodiments or aspects of the invention.

Embodiments of the invention will now be described in more detail, by way of example only, with reference to the accompanying figures.

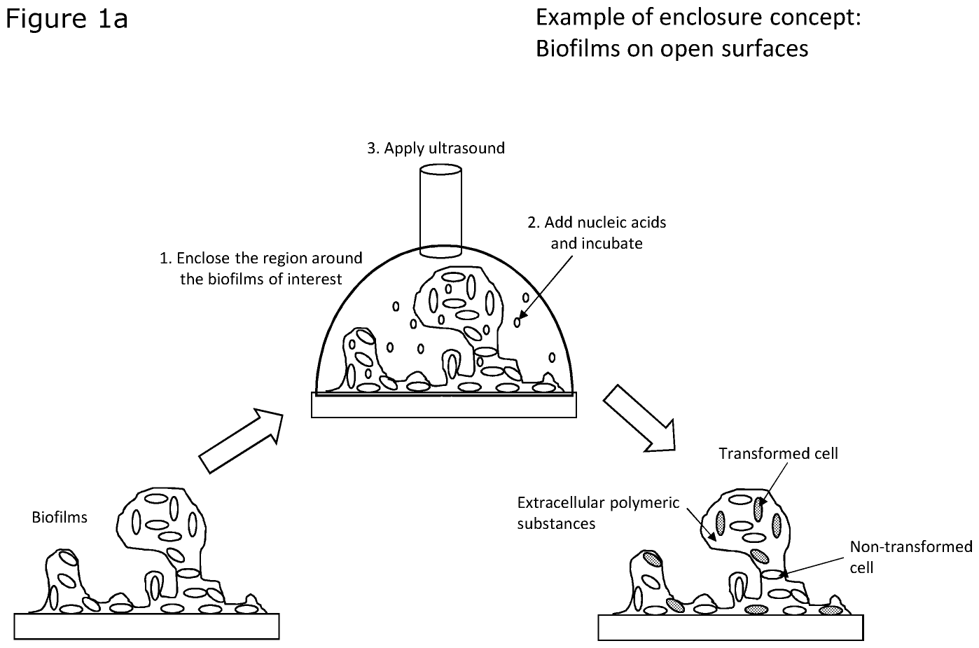

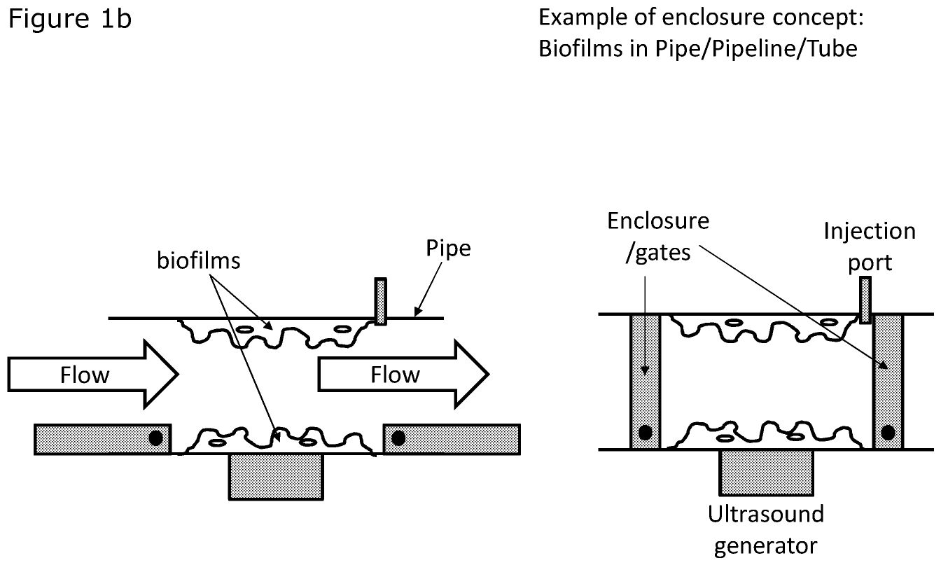

FIG. 1—(A) Ultrasound-based transformation of biofilm growing on open surface enclosed with barrier in the form of a movable cup. (B) Ultrasound-based transformation of biofilm growing within a pipe enclosed with movable barriers at both ends. (C) Ultrasound-based transformation of biofilm growing within a tank enclosed with movable barrier.

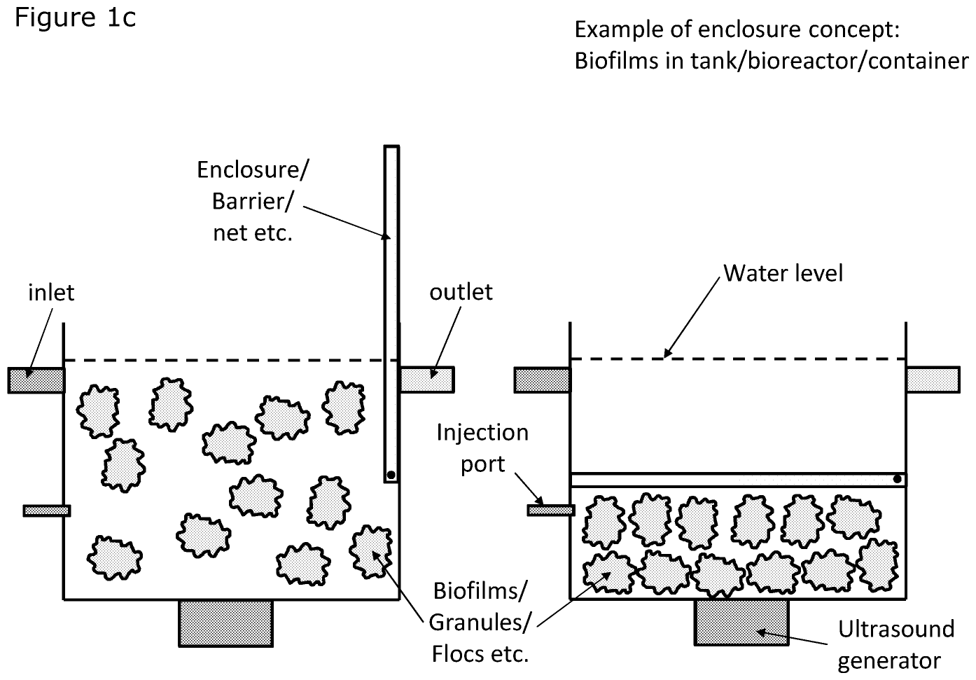

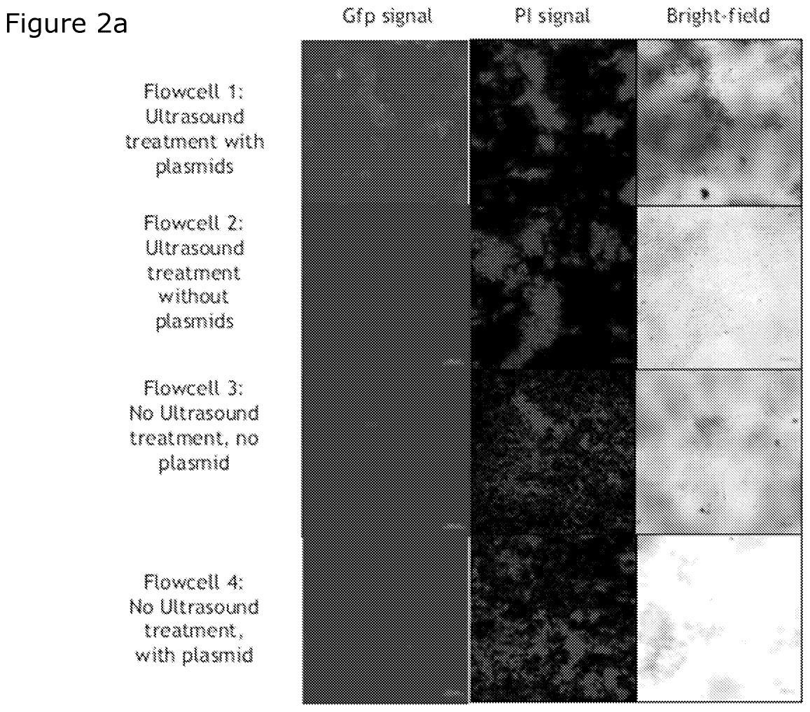

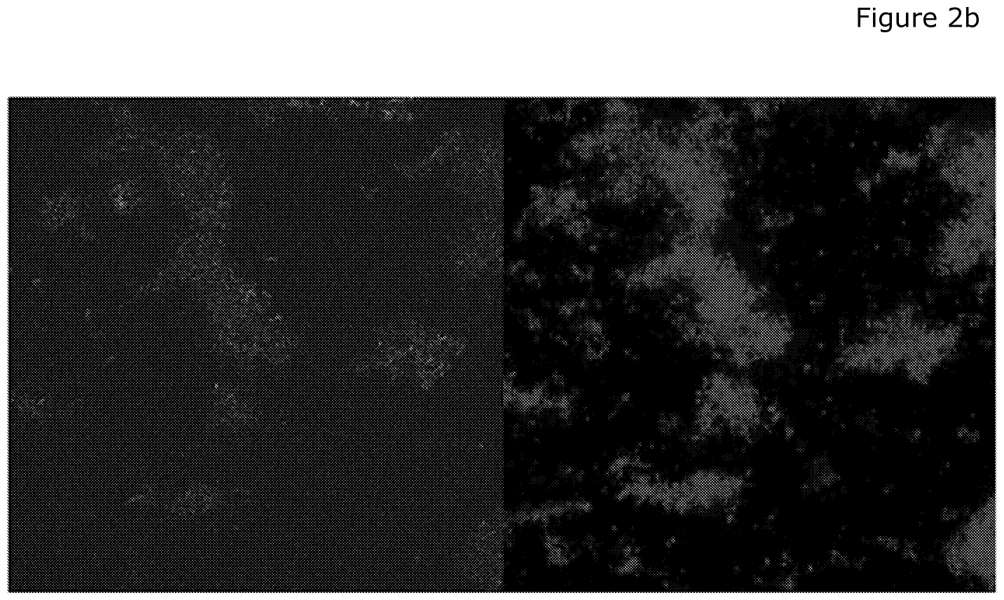

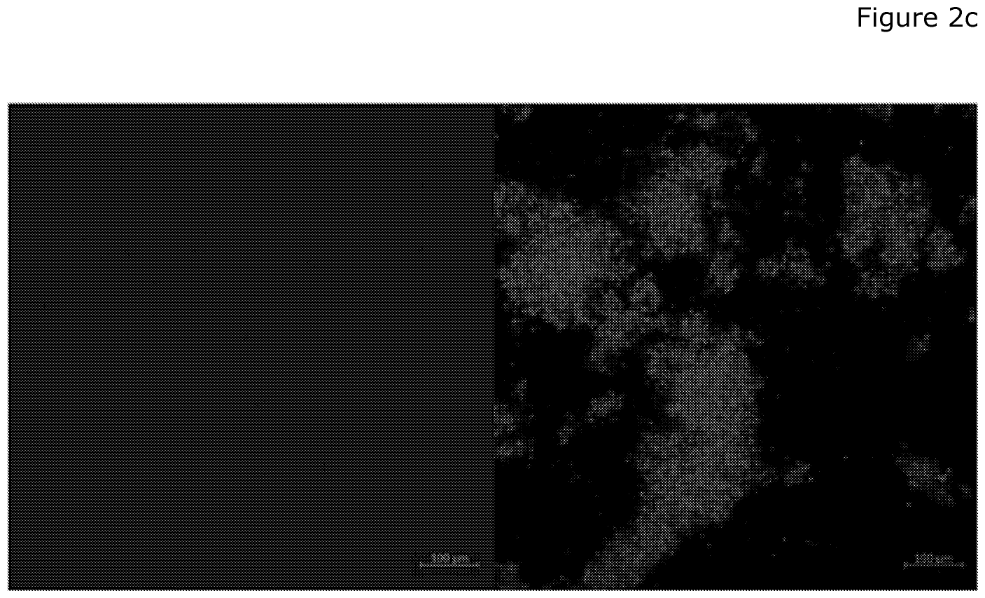

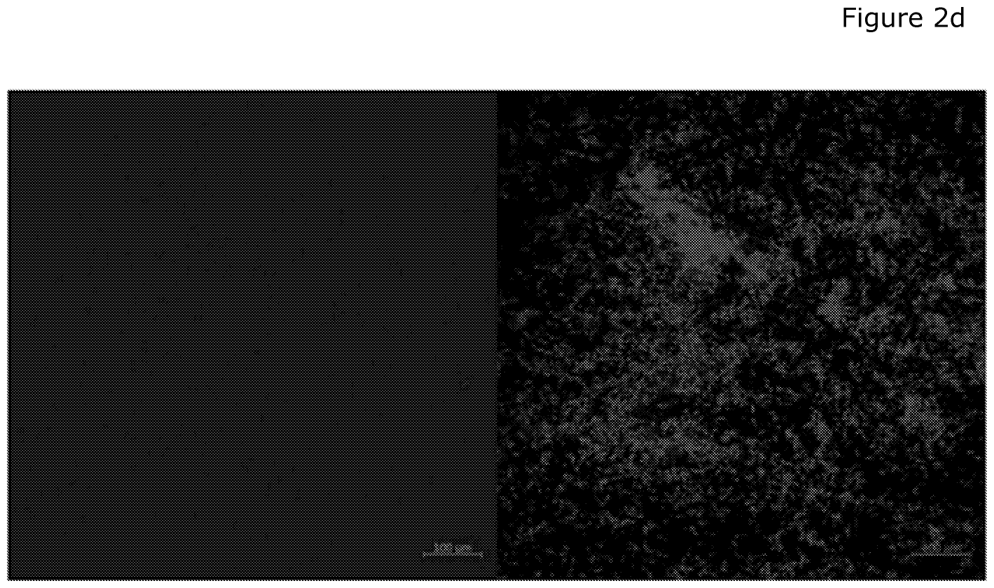

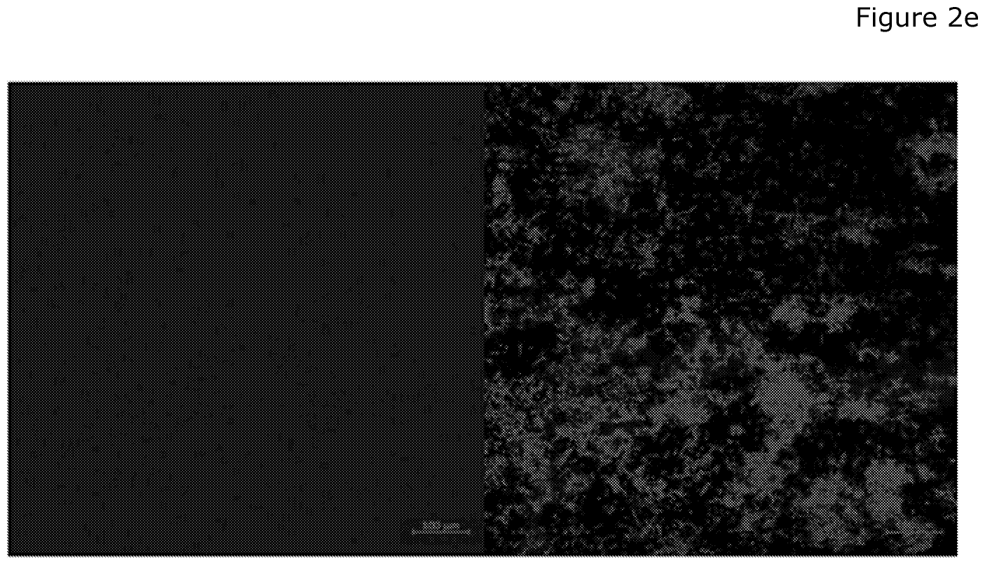

FIG. 2—(A) Experiment demonstrating the transformation of biofilms in flowcells under different experimental conditions. (B) GFP signal (left) and PI signal (right) after 10 s of 40 kHz ultrasound treatment and ˜1 ng/mL plasmid. (C) PI signal (right) and lack of GFP signal (left) after 10 s of 40 kHz ultrasound treatment and no plasmid. (D) PI signal (right) and lack of GFP signal (left) after no ultrasound treatment and no plasmid. (E) PI signal (right) and lack of GFP signal (left) after no ultrasound treatment and ˜1 ng/mL plasmid. All experiments were carried out with the addition of 0.3 mL CaCl₂ which contained the plasmid, where relevant.

FIG. 3—Waste bottles following flowcell experiments. Growth is only seen in the waste bottle from the flow cell treated with ultrasound and plasmid.

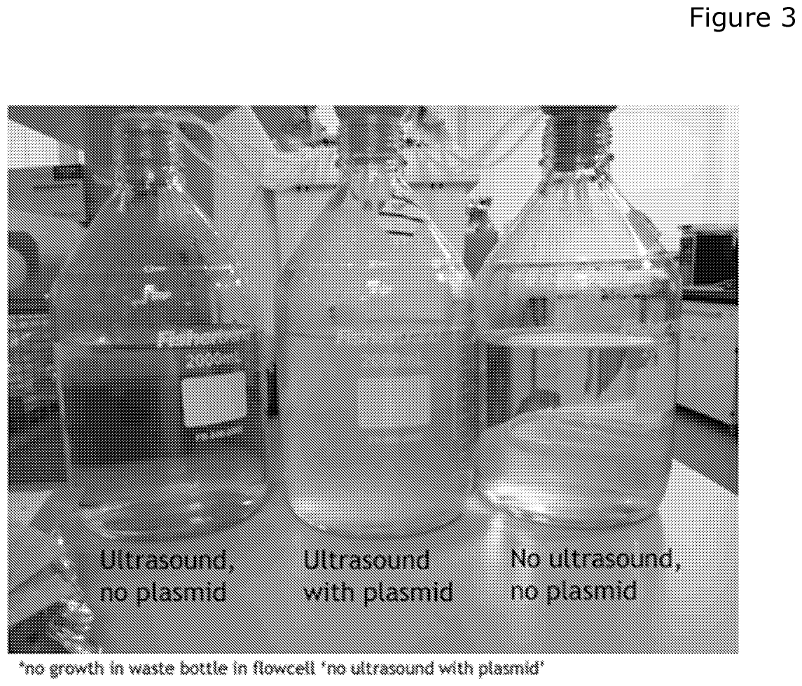

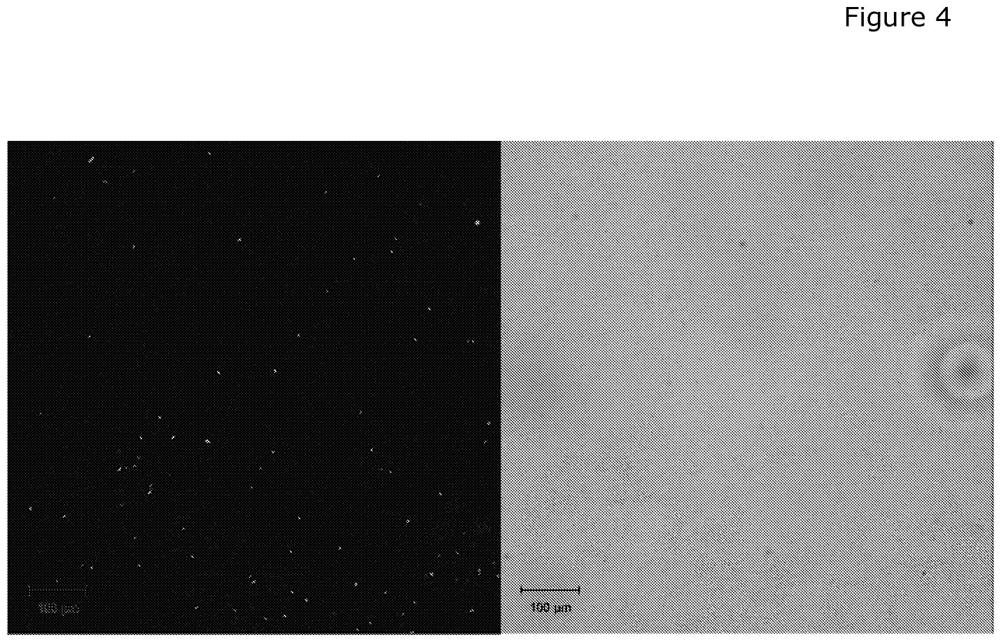

FIG. 4—0-5 h after transformation with ultrasound. Within 5 h, some transformed cells begin to express sfGFP but with limited cell replication. Left image, white are cells that are transformed and have expressed sfGFP.

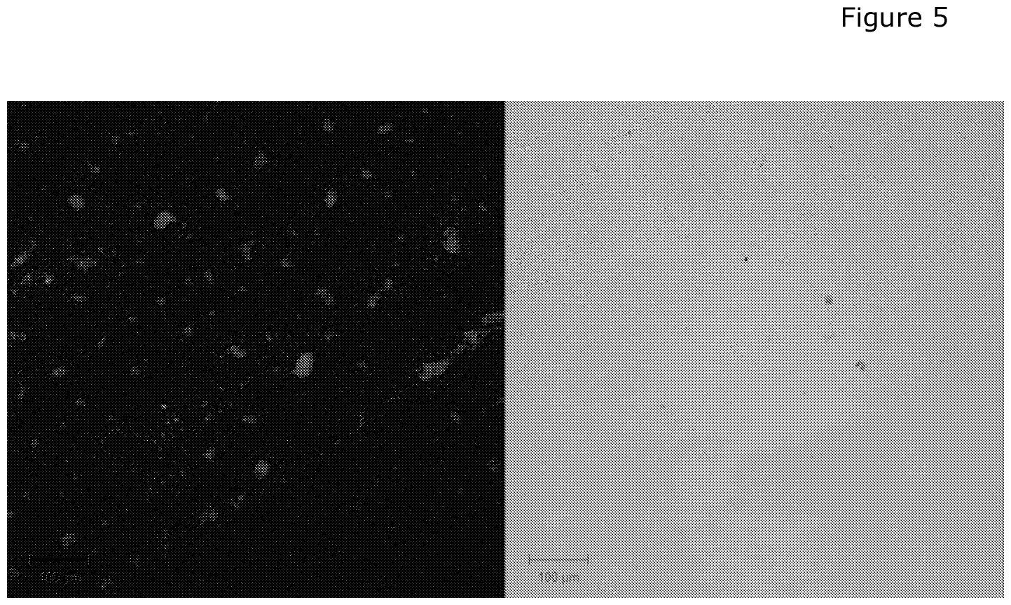

FIG. 5—24 h after transformation with ultrasound. More transformed cells are expressing sfGFP (left image; white) and possibly growth of transformed cells into micro-colonies in media+Km.

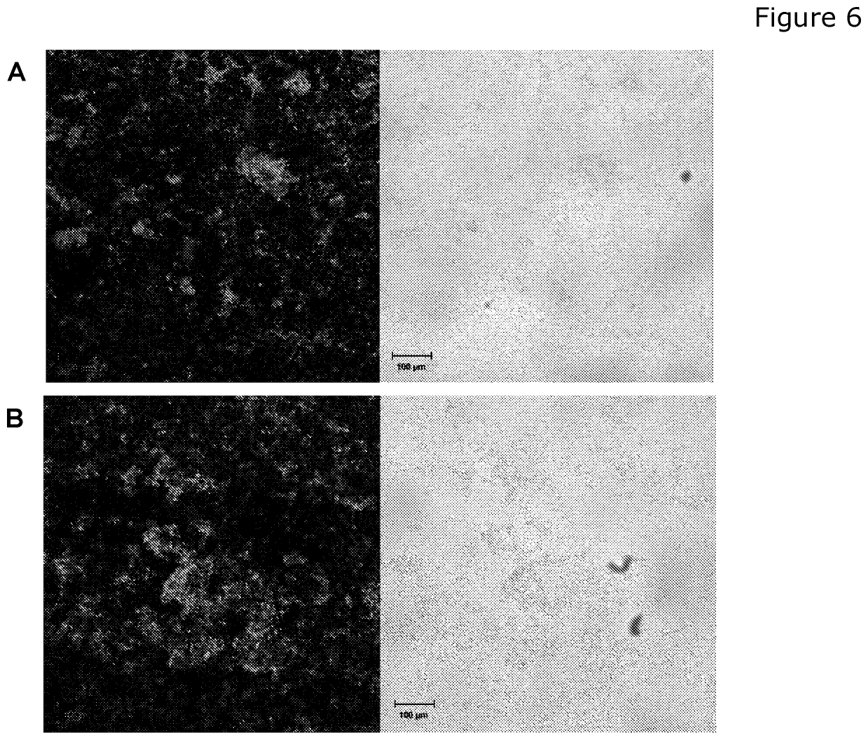

FIG. 6—48 h after transformation with ultrasound. (A) More cells expressing sfGFP and possibly growth of transformed cells in media+Km (B) More cells expressing sfGFP and growth of transformed cells in media+Km. sfGFP expression is shown as white in the left images.

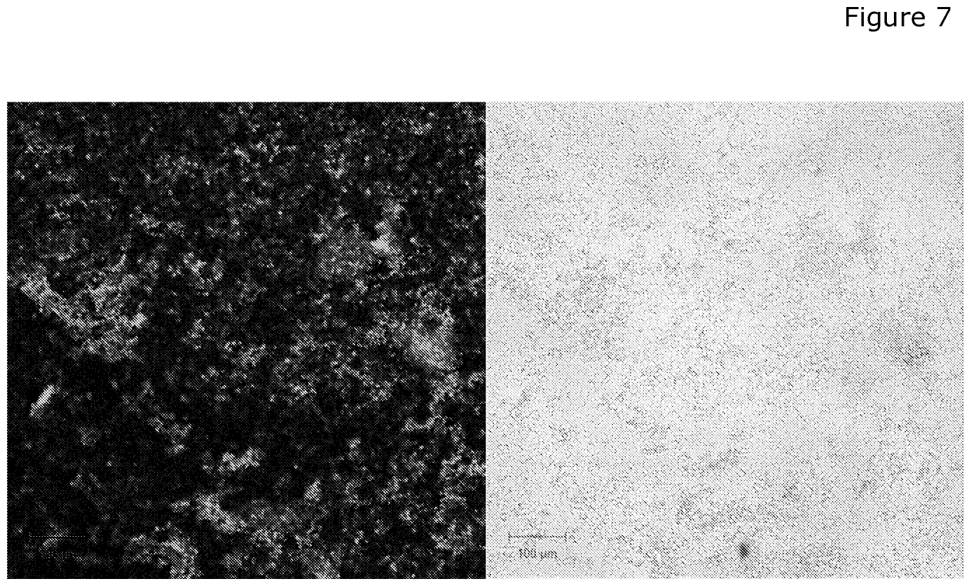

FIG. 7—5 days after transformation with ultrasound. Transformed cells growing among non-transformed dead or persister cells. sfGFP expression is shown as white in the left images and identifies transformed cells.

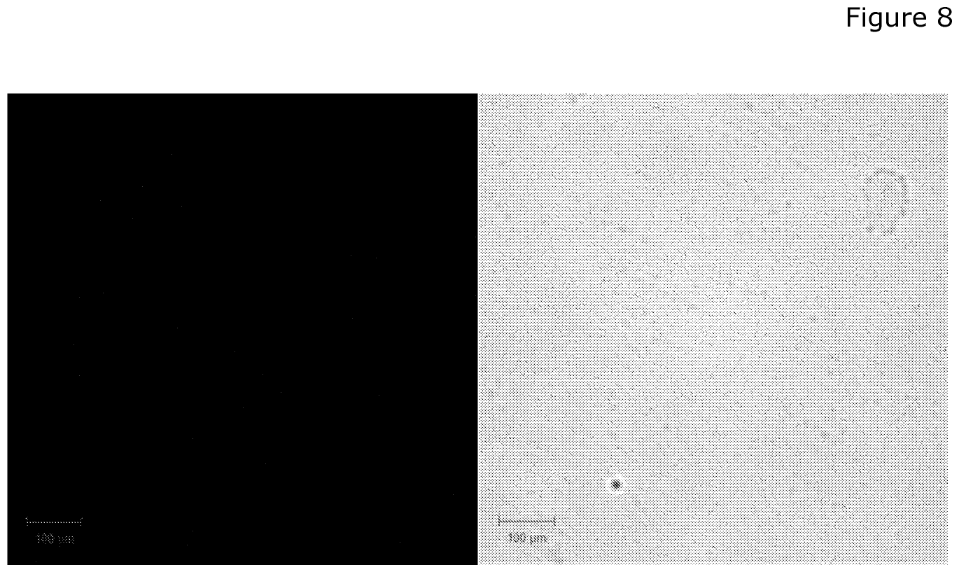

FIG. 8—5 days after transformation without ultrasound. A layer of non-transformed dead or persister cells with no expression of sfGFP in media+Km.

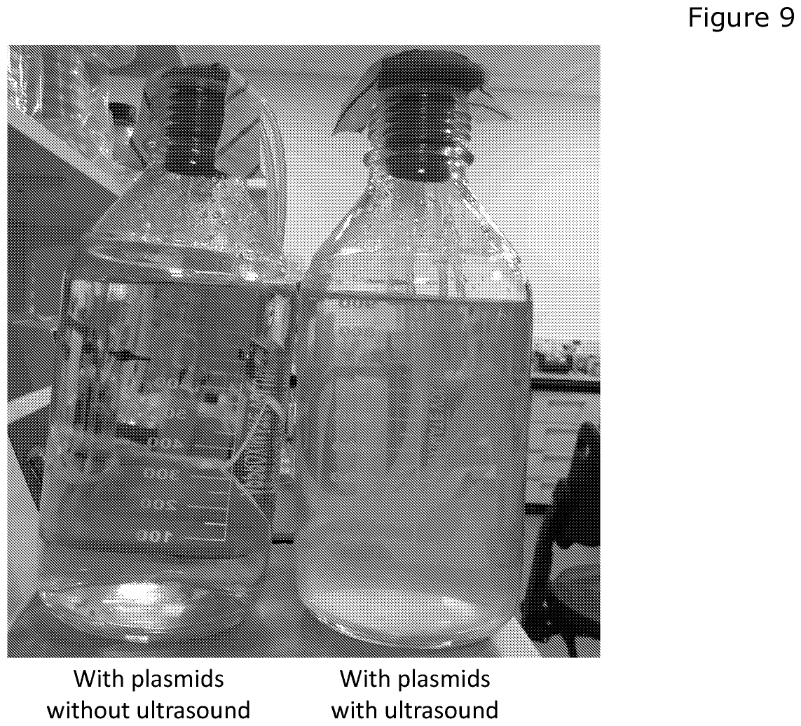

FIG. 9—Media contains antibiotics to select for transformed cells over non-transformed cells within the biofilm. No growth detected in the waste media bottle of the flow cells with plasmids but without ultrasound treatment. This is a proof-of-concept that native biofilms are able to acquire new properties (e.g. antibiotic resistance).

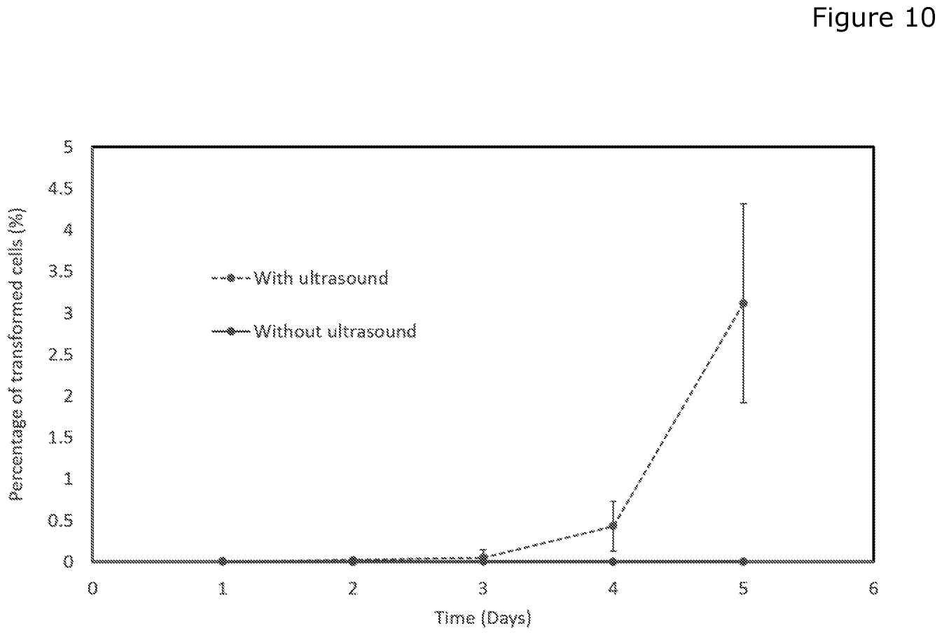

FIG. 10. Percentage of transformed cells in biofilms that are treated with or without ultrasound. Biofilms were incubated with 1 ng/mL plasmid and 50 mM CaCl₂ for 10 minutes prior to treatment.

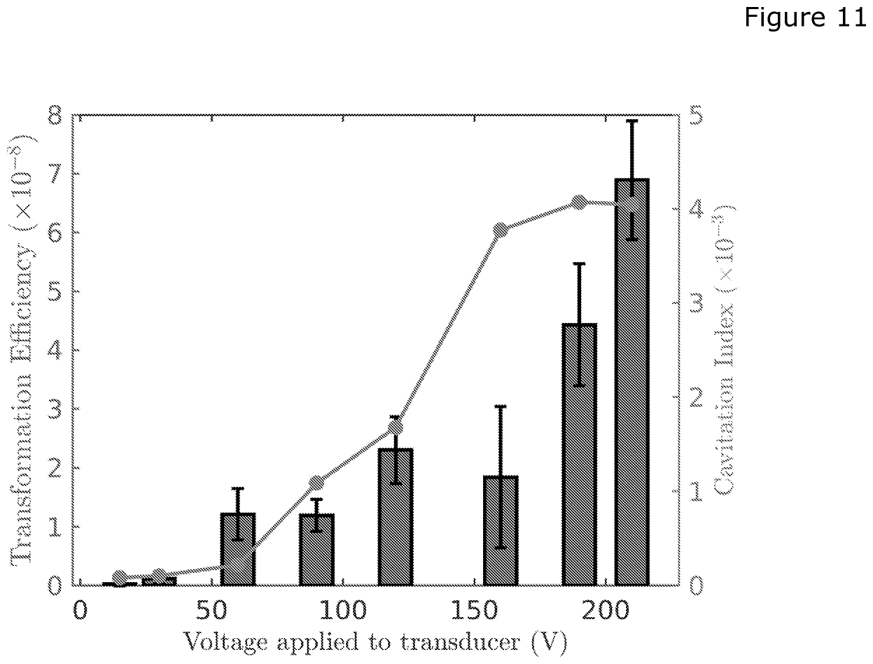

FIG. 11. Data on transformation efficiency and inertial cavitation signal vs voltage. Voltage applied to the ultrasound system determines the level of cavitation activity, as indicated by broadband noise and expressed as the cavitation index. From the graph, cavitation index and transformation efficiency followed the same trend where higher cavitation is accompanied with higher transformation efficiency, which is an indication that inertial cavitation is involved in transformation of cells in biofilms.

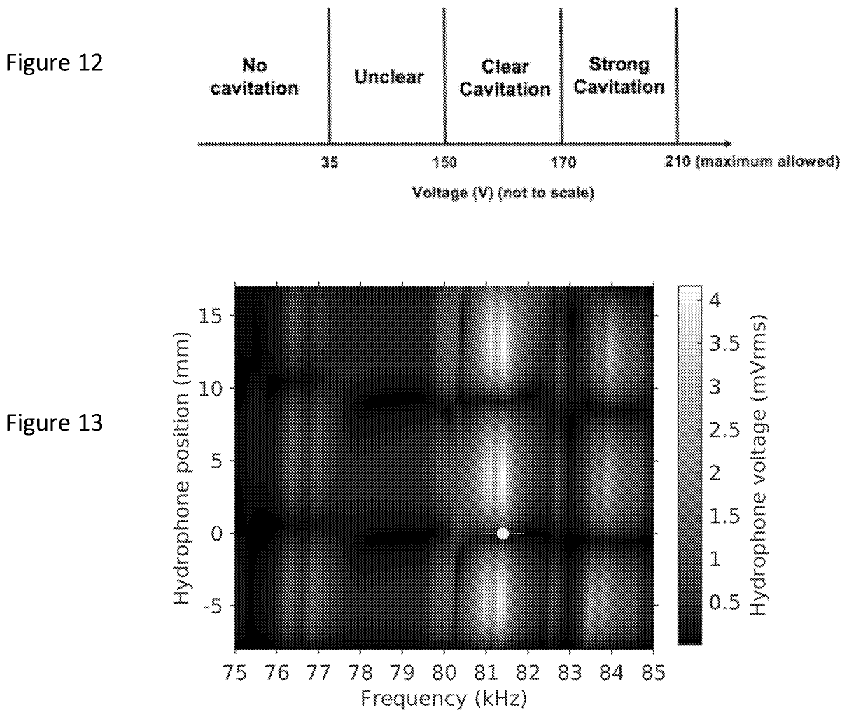

FIG. 12. Qualitative description of cavitation level at different voltages.

FIG. 13. Plot of frequency response of the system as a function of hydrophone position in the resonator (depth). The hydrophone was positioned at an acoustic node for cavitation noise measurements at 81.4 kHz (indicated by yellow ‘+’ in plot). The top (water surface) of the resonator is located at −8 mm.

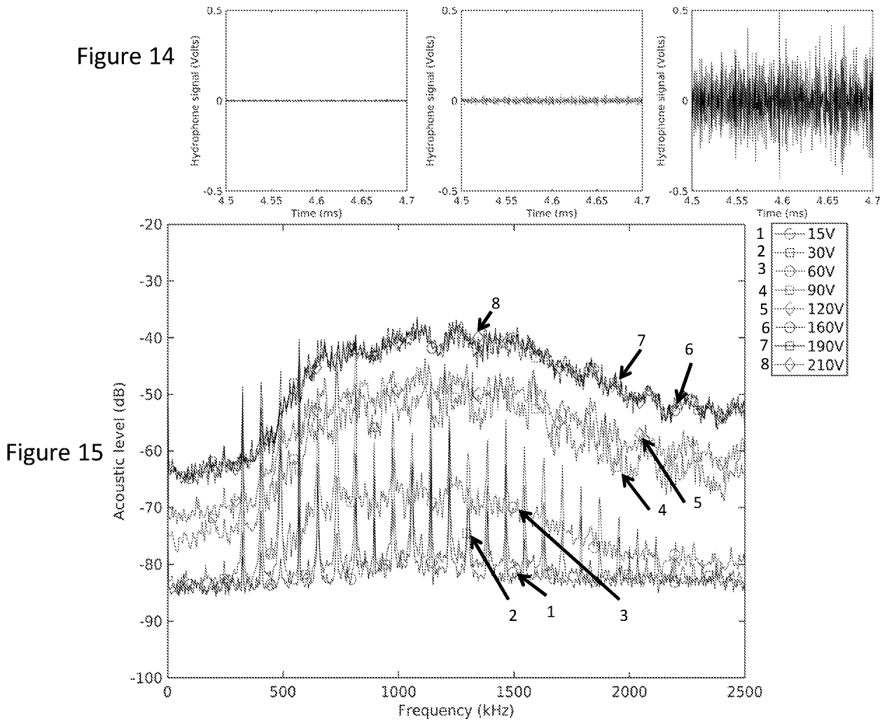

FIG. 14. Examples of cavitation noise measured at 30V, 60V, and 190V. Left panel=no cavitation, Middle panel=unclear, and Right panel=clear cavitation or strong cavitation.

FIG. 15. Frequency spectrum of cavitation noise measured at drive levels from 15-210 V. The numbers correspond to regions qualitatively labeled ‘no cavitation’ (1 and 2), ‘unclear’ (3, 4 and 5), and ‘clear cavitation’ or ‘strong cavitation’ (6, 7 and 8). The low end roll-off is due to the high-pass filter.

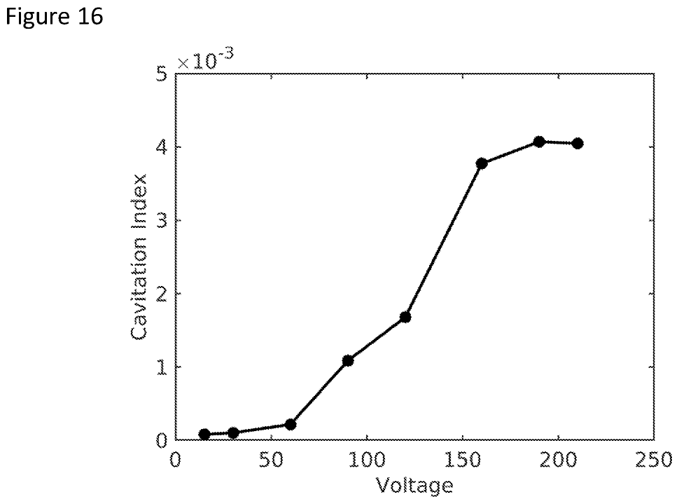

FIG. 16. Plot of the cavitation index as a function of drive voltage.

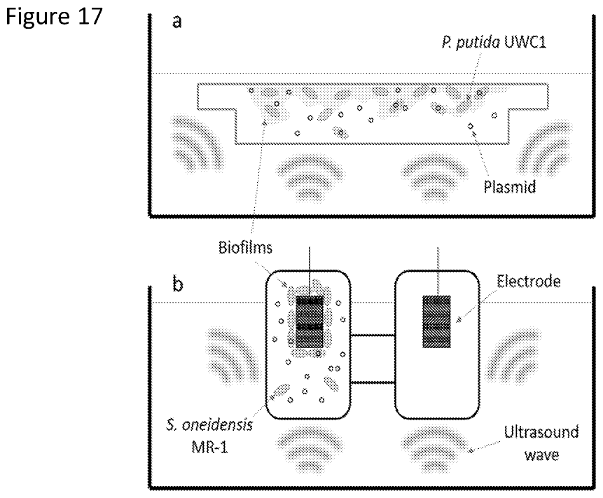

FIG. 17. Schematic diagram of ultrasound-based DNA delivery (UDD) into bacterial cells of mature biofilms established in a) microfluidic flow cells and b) microbial fuel cell (MFC). Ultrasound treatments were applied in a commercially available 40 kHz ultrasound clean bath. Diagram is not drawn to scale.

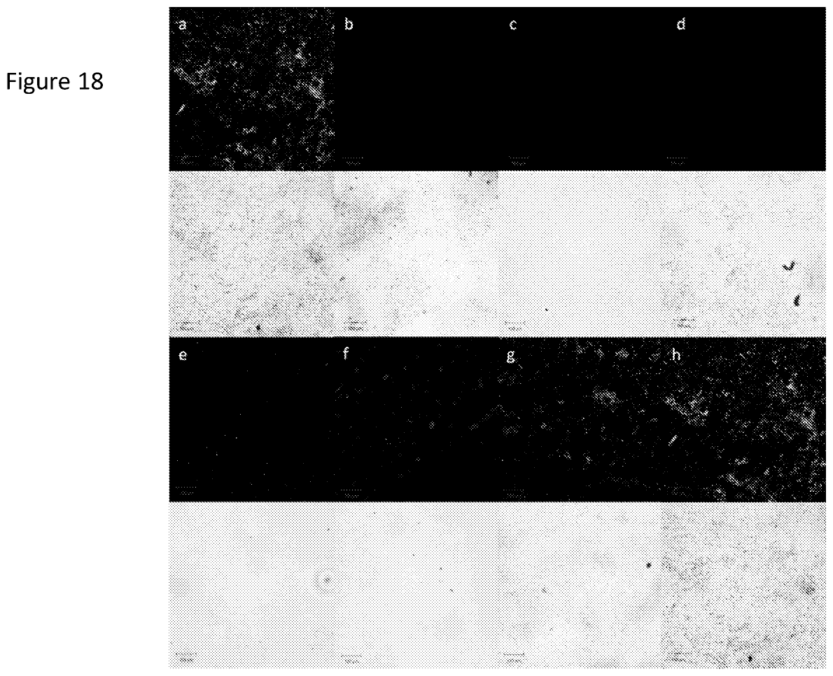

FIG. 18. Green fluorescence signal and bright field imaging for biofilm samples after 120 h with a) both addition of plasmid and ultrasound treatment (+P/+U), b) only addition of plasmid (+P/−U), c) only ultrasound treatment (−P/+U) and d) no plasmid and no ultrasound treatment. Green fluorescence signal and bright field imaging for biofilm samples with both addition of plasmid and ultrasound treatment after e) 5 h, f) 24 h, g) 48 h, h) 120 h.

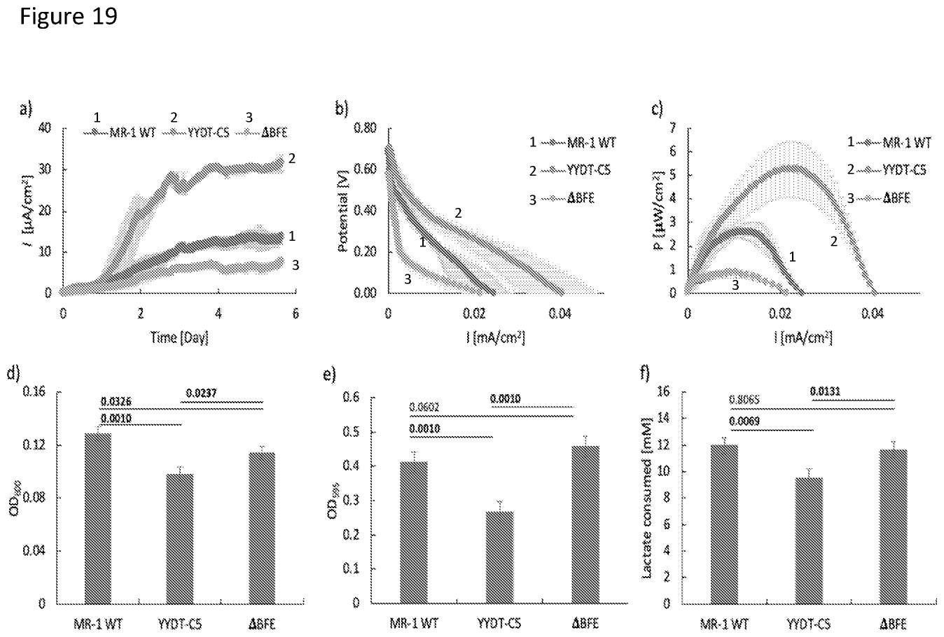

FIG. 19. a) Current density (I) over time, b) Polarisation curve (Current density vs. Potential) and c) Power curve of MFC reactors with S. oneidensis MR-1 WT (1), MR-1/YYDT-C5 mutant (2) and MR-1 Δbfe strains (3) with 20 mM sodium lactate as sole carbon source. Measurement was conducted via Linear Sweep Voltammetry (E_{begin}=0.8V, E_{end}=0.0V, Eₛₜₑₚ=0.1V, scan rate=0.1 mV/s) once steady-state current was achieved over 1 kΩ resistor (˜day 6). Error bars represent standard deviation of triplicate measurements. d) Optical density at 600 nm (OD₆₀₀) of anodic culture of MFC reactors utilising S. oneidensis MR-1 WT, MR-1/YYDT-C5 mutant and MR-1 Δbfe strains with 20 mM sodium lactate as sole carbon source. Measurement was done using 1 cm cuvette (1 mL sample size). e) Biofilm quantification using crystal violet assay: optical density at 595 nm (OD₅₉₅) of cell-bound crystal violet solution from anodic biofilm cells of the MFC reactors. f) Amount of lactate consumed by each reactor. Produced metabolites were mainly acetate, with succinate and pyruvate in trace amounts (data not shown). Measurements in Figure d), e) and f) were done at the end of MFC experiment (day 13). Error bars represent standard deviation of triplicate measurements. P values on top of the bars denote differences between sample pairs based on nested mixed-factor ANOVA test followed by Tukey's HSD post hoc test. P values showing statistically significant (p<0.05) differences are presented in bold.

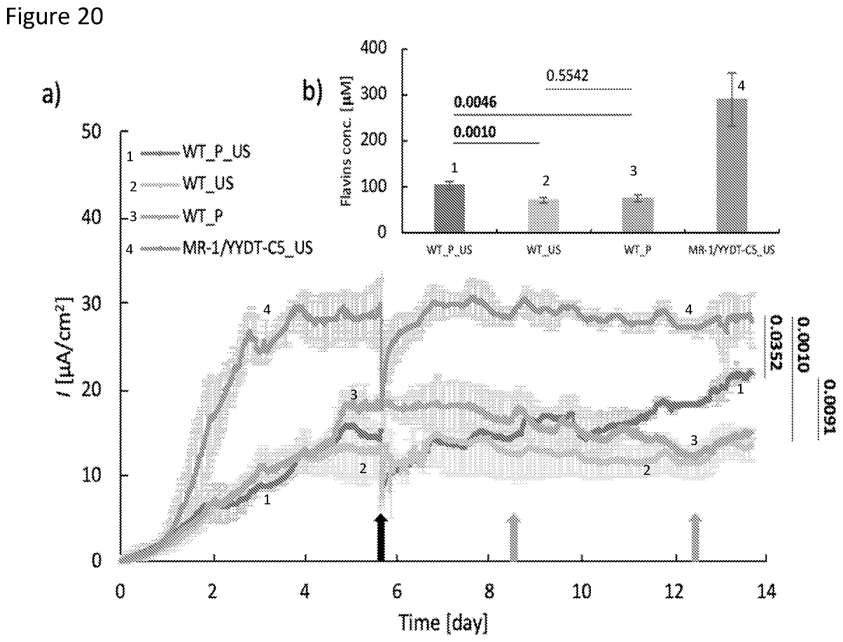

FIG. 20. a) Current density (I) of double-compartment MFC reactors running at 1 kΩ load with 20 mM initial concentration of sodium lactate; b) extracellular flavins concentration of MFC reactors after 14 days of operation. Four different type of reactors: MR-1/YYDT-C5 strain (MR-1/YYDT-C5_US, 4), MR-1 WT with addition of plasmid and ultrasound treatment (WT_P_US, 1), MR-1 WT with only ultrasound treatment (WT_US, 2), and WT with only addition of plasmid (WT_P, 3). Ultrasound was performed for 30 s on day 6 (arrow—A) for appropriate MFC setups. On day 9, kanamycin (10 μg/mL) and 10 mM of additional lactate was added into each reactor (arrow—B). On day 13, additional kanamycin was added to reach final concentration of 50 μg/mL (arrow—C). Shaded regions represent standard deviations of triplicate measurements. P values on top of the bars were calculated for the last day of measurement and denote differences between sample pairs based on nested mixed-factor ANOVA test followed by Tukey's HSD post hoc test. P values showing statistically significant (p<0.05) differences are presented in bold.

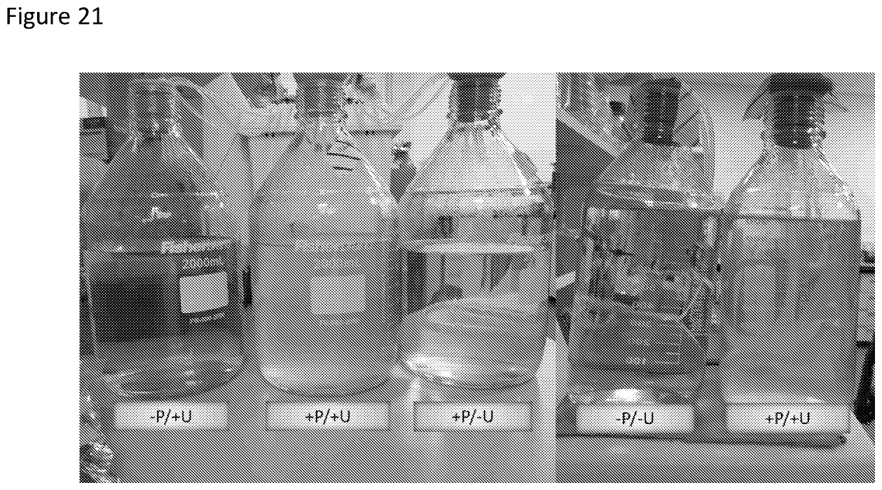

FIG. 21. The growth media outputs of the flow cells under various conditions: presence of plasmids with ultrasound treatment (+P/+U), presence of plasmid without ultrasound treatment (+P/−U), absence of plasmid with ultrasound treatment (−P/+U), and absence of both plasmid and ultrasound treatment (−P/−U).

### EXAMPLES

### Example 1

### Proof of Concept Study

Biofilms were grown in four flow-cells for 72 hours. Each flow-cell received a different treatment to determine the effectiveness of ultrasound treatment in the transformation of intact biofilms. Plasmids used in the experiment were encoded with green fluorescent protein (gfp) and kanamycin resistance. The flow-cells were treated as follows:

Flow-cell 1: 0.3 mL CaCl₂ containing ˜1 ng/mL plasmid was added to the flow-cell, followed by 10 seconds of 40 kHz ultrasound treatment.

Flow-cell 2: 0.3 mL CaCl₂ containing no plasmid was added to the flow-cell, followed by 10 seconds of 40 kHz ultrasound treatment.

Flow-cell 3: 0.3 mL CaCl₂ containing no plasmid was added to the flow-cell. No ultrasound treatment was given.

Flow-cell 4: 0.3 mL CaCl₂ containing ˜1 ng/mL plasmid was added to the flow-cell. No ultrasound treatment was given.

Following treatment, kanamycin was added to the growth media to select for transformed cells. To further confirm successful transformation, all flow-cells were stained with PI and imaged under confocal laser scanning microscope, using the same settings for each flowcell. Only flow-cell 1, treated with both the plasmid and ultrasound showed expression of gfp (FIG. 3). Waste from the flow-cells was collected in waste bottles which were assessed for cell growth following the experiment. Only the waste bottle from flow-cell 1, treated with both the plasmid and ultrasound, showed evidence of cell growth (FIG. 4), demonstrating that cells in flow-cell 1 had resistance to kanamycin.

### Example 2

### gfp Expression and Cell Growth After Transformation

Biofilm was grown in flow-cells for 3 days to reach maturation before 1 ng/μl plasmid (pBBR1.MCS_sfGFP, harbouring superfolder green fluorescence protein and kanamycin (Km) resistance gene) in 50 mM CaCl₂ was introduced to the flow-cells and incubated for 10 minutes. The length of incubation is dependent on the thickness of the biofilm. After the incubation period, flow-cells were treated with 42 kHz ultrasound for 10 s. 1/10 Luria-Bertani broth (LB) was reintroduced onto the flow-cell at 20 mL/h for 2 hours, then replaced with 1/10 LB with Km (5 μg/mL) at 20 mL/h for 5 days.

FIG. 4 demonstrates the initial transformation of cells which begin to express sfGFP within 5 hours of transformation. Increasing levels of sfGFP expression is identified over the subsequent 48 hours and after 5 days non-transformed dead or persister cells can be identified among the transformed sfGFP cells in the biofilm (FIGS. 5-7). Biofilms that were exposed to the plasmid, but not the ultrasound treatment, showed no evidence of transformation after 5 days (FIG. 8) and no growth was seen in the waste bottles connected the flow-cell (FIG. 9). FIG. 10 shows percentage of transformed cells over total cells in biofilm after ultrasound treatment with and without plasmids.

### Example 3

### Mechanism of Ultrasound-Based Transformation in Biofilms

Transformation efficiency of cells were measured under different voltage applied to the ultrasound transducer producing different amount/strength of cavitation (cavitation index). There is a strong correlation between cavitation index and transformation efficiency, which is an indication that cavitation is one of the key mechanisms in ultrasound-based transformation in biofilms. (FIG. 11).

### Example 4

### Cavitation Noise Measurements in 80 kHz Resonator

Introduction

The purpose was to conduct a quantitative measurement of cavitation noise in an 80 kHz resonator system and complement the description shown in FIG. 12.

Equipment Used

Function generator—LeCroy Wavestation 2022

Power Amplifier—Amplifier Research 75A250A

Hydrophone—Dynasen CA1136-6″ with CA1146 cable

Oscilloscope—LeCroy LT344

Voltage probe—LeCroy PP006 10:1

Current probe—Pearson

Methods

The transducer and horn (with o-ring) were inserted completely into the resonator leaving 3.5-4 cm space above the transducer to the top of the cylindrical resonator. The resonator was filled with degassed water at room temperature.

A function generator (LeCroy Wavestation 2022), attenuator (−20 dB; Digi-Key 367-1120-ND) and power amplifier (Amplifier Research 75A250A) were used to excite the transducer at frequency near the nominal design resonance of the system (80 kHz). The drive voltage (10:1 Probe PP006, LeCroy) and current (1 Volt/Ampere; Pearson 6016) were monitored on the oscilloscope (CH1 and CH2 respectively; LeCroy LT344). A detector consisting of a 0.05″ diameter×0.020″ thick PZT disc (Dynasen CA1136-6″ with CA1146 cable) was used as a hydrophone to measure the in-situ acoustic pressure and cavitation noise. The hydrophone signal was fed to an active high-pass filter (Krohn-Hite 34A; −24 dB/octave) with cutoff frequency (−3 dB) set to 1 MHz and +20 dB output gain and digitized on the oscilloscope (CH4; 5 MS/s, 20 us/div, 4.5 ms trigger delay).

The method of using a hydrophone in direct contact with the water was selected for the purpose of producing a quick and reliable measurement of cavitation noise. It should be noted that the presence of the hydrophone in the acoustic field could affect the resonance behavior of the system and the cavitation threshold. Preliminary experiments suggested this method resulted in a resonance frequency/threshold consistent with the same that was observed when the hydrophone was removed, however this effect was not quantified. The hydrophone was positioned at an acoustic node to minimize the effect of the standing wave acoustic signal at the fundamental frequency on the detection of the cavitation noise. The system was tuned to the optimal frequency ‘by-ear’, such that cavitation noise could just be detected at moderate drive level (120 V) but such that changing the frequency up or down (eg. by 0.1 kHz caused the cavitation noise to turn off). The optimal frequency was found to be 81.4 kHz. In subsequent trials it was observed that the electrical current to the transducer went through a phase change in the frequency range 81.3-81.4 kHz, confirming this was a resonance frequency of the system. A plot of the frequency response of the system as a function of hydrophone position was obtained as a sanity check and is shown in FIG. 13. The frequency spectrum was obtained using spectrum analyzer (HP3585A) with tracking generator and drive voltage set at low level (+0 dBm output→−20 dB attenuator→Power Amplifier at lowest gain) in conjunction with a robotic positioning system (Velmex) and automated MATLAB script.

Results

Cavitation Noise

The measured acoustic signal is shown in FIG. 14 and the frequency spectrum is shown in FIG. 15. The peaks in the spectrum at low drive levels occur at multiples of the drive frequency and could be due to harmonics generated by nonlinearities in the mechanical system (eg. similar mechanism to the vibrations which cause the audible ‘system noise’) or possibly nonlinearities in the PZT disc sensor such as radial modes or strain saturation. Audible observations included a high-pitch ringing ‘system noise’ audible at 15V, 30V and 60V. At 90V and 120V cavitation noise was intermittent and ‘system noise’ audible but not necessarily louder than at lower levels. At 160V, 190V and 210V strong cavitation noise was observed and the ‘system noise’ was mostly masked by the cavitation noise.

A cavitation index can be used to aid interpretation of the cavitation noise data. The cavitation index is defined here as the arithmetic mean of the values of the frequency spectrum between 100 kHz and 2.5 MHz and quantifies the broadband noise generated by microbubble collapse (Sabraoui 2011, Inserra 2014). The cavitation index has been plotted in FIG. 16 on a linear scale by taking the anti-logarithm.

### REFERENCES

Cochrane, J. An acoustic cavitation reactor for quantifying the effect of cavitation on cell suspensions. M. Eng. Report, Wadham College, University of Oxford, 2018.

Inserra C, Labelle P, Der Loughian C, Lee J-L, Fouqueray M, Ngo J, Poizat A, Desjouy C, Munteanu B, Lo C-W, Vanbelle C, Rieu J-P, Chen W-S, Béra J-C. Monitoring and control of inertial cavitation activity for enhancing ultrasound transfection: The SonInCaRe project. IRBM 2014; 35:94-99.

Sabraoui A, Inserra C, Gilles B, Bera J C, Mestas J L. Feedback loop process to control acoustic cavitation. Ultrason Sonochem 2011; 18:589-94.

### Example 2

### Biofilm Engineering: Applications of Ultrasound-Based DNA Delivery (UDD) Toward In-Situ Bacterial Transformation in Established Biofilms

### SUMMARY

The ability to augment native and established biofilm communities has long been a goal but the technology remains elusive. In this study, we explored the potential of the ultrasound-based DNA delivery (UDD) system to induce in-situ plasmid uptake by non-competent bacterial cells of established biofilms). DNA fragments (i.e. plasmids) containing genes coding for super folding green fluorescence protein (sfGFP) and flavin synthesis pathway were introduced into established bacterial biofilms cultured in microfluidic flow and microbial fuel cells (MFC) respectively employing UDD. Phenotypic signals of successful bacterial transformation in established biofilms were observed, where UDD-treated P. putida UWC1 biofilms developed green fluorescence signal in flow cells and UDD-treated S. oneidensis MR-1 biofilms generated higher bioelectricity production in MFC compared to the control group. The effects of UDD were amplified in subsequent growth under selective pressure. This study reports on a scalable method developed for the first time towards genetic and phenotypic control of established biofilms for environmental, industrial and medical applications.

Here more direct and immediate ways of augmenting biofilms central to the functioning of many engineering systems (e.g. bioreactor) have been developed. In the previous study, successful applications of low frequency 40 kHz ultrasound for transferring plasmids into three different bacterial species in their planktonic states were achieved with a high rate (9.8±2.3×10⁻⁶ per mg) of gene uptake¹⁷. An aim for this study is to determine the potential of ultrasound-based DNA delivery (UDD) for biofilms in microfluidic flow cells and MFCs, while demonstrating the scalability of this technology.

Results

Ultrasound-Mediated DNA Delivery (UDD) in Flow Cell Biofilms

A flow cell system was set up to examine the effectiveness of UDD to transfer plasmids to established biofilm (FIG. 17a). After 3 days of incubation UWC1 biofilms were established in flow cells and treated under 4 conditions: with both addition of plasmid and ultrasound treatment (+P/+U), with only ultrasound treatment (−P/+U), with only addition of plasmid (+P/−U), and without addition of plasmid nor ultrasound treatment (−P/−U). The frequency of ultrasound was 40 kHz. The ultrasound treatment time was 10 s. The plasmid pBBR1MCS-2_Plux_sfGFP (Table 1) contained a broad host pBBR1 backbone and the superfold gfp gene was fused with a constitutive promoter. Five hours after addition of plasmid and ultrasound treatment, small spots of green fluorescence signal were observed across the +P/+U biofilm samples (FIG. 18e), presumably produced by UWC1 cells that were successfully transformed. All samples were subjected to longer incubation period under constant flow of growth media containing kanamycin to exert selection pressure for transformed cells over non-transformed ones. Clusters of green fluorescence signals formed after 24 hours (FIG. 18f) in +P/+U biofilm samples and the area of green fluorescence signal continued to expand over 48 hours (FIG. 18g) and 120 hours (FIG. 18h). Over the same period, samples without either ultrasound treatment (+P/−U, FIG. 2b) or the addition of plasmid (−P/+U, FIG. 2c) or both (−P/−U, 2d) showed no signs of sfGFP production. The growth media outputs of the flow cell were also examined and only +P/+U samples showed ‘cloudiness’ indicating bacterial growth, whilst there no signs of any growth in the other samples (FIG. 21). This suggests that ultrasound mediated DNA delivery transfer plasmid into biofilm had occurred.

The samples from the established biofilms in four treatments were taken and cultured in LB medium with kanamycin. Only the samples from +P/+U treatment can grow but the samples from other three controls were unable to grow in the presence of kanamycin. The plasmids were extracted and the sequence confirmed that the recovered plasmid was pBBR1MCS-2_Plux_sfGFP. These results demonstrated that both ultrasound and plasmid are required for bacterial transformation to take place, and provided the proof-of-concept where UDD can be deployed towards in-situ bacterial transformation within established biofilms.

Flavin Electron Shuttles are the Dominant Mechanism of Electron Transfer in Shewanella oneidensis MR-1

The biofilms of Shewanella oneidensis MR-1 wild type (WT), MR-1 Δbfe (knockout of bfe gene for bacterial flavin adenine dinucleotide [FAD] exporter)¹⁸ and MR-1/YYDT-C5 (MR-1 with plasmid pYYDT-C5 containing the entire flavin biosynthesis gene cluster ribADEHC cloned from Bacillus subtilis)¹⁹ were established in the MFC system. The steady-state current density generated by the MR-1 WT reached 13.7±0.3 μA/cm², compared to 7.6±0.1 μA/cm² for the MR-1 Δbfe and 31.5±1.8 μA/cm² for the MR-1/YYDT-C5 mutant (p<0.05) (FIG. 19a; Table 2). After 13 days of operation, MR-1/YYDT-C5 showed highest current density vs. potential as compared to MR-1 WT and MR-1 Δbfe (FIG. 19b). The maximum power output of the MR-1 WT was 2.61±0.35 μW/cm², compared to 0.83±0.19 μW/cm² from the MR-1 Δbfe, whilst MR-1/YYDT-C5 reached 5.25±1.18 μW/cm² (p<0.05) (FIG. 19c). The OD₆₀₀ of anodic culture in the MR-1 WT reactors reached 0.129±0.005, whilst that of MR-1/YYDT-C5 and MR-1 Δbfe peaked at a density of 0.098±0.005 and 0.114±0.005 respectively (FIG. 19d). The OD₅₉₅ of cell-bound crystal violet solution from anodic biofilm cells of the MR-1 WT was found to be 0.411±0.030, and 0.267±0.031 for MR-1/YYDT-C5 and 0.458±0.030 for MR-1 Δbfe (FIG. 19e). This translated into the number of attached cells in the biofilm as (2.74±0.18)×10⁵/cm² for MR-1 WT, (1.78±0.24)×10⁵/cm² for MR-1/YYDT-C5 and (3.05±0.20)×10⁵/cm² for MR-1 Δbfe. Consumption of lactate in MR-1 WT, MR-1/YYDT-C5 and MR-1 Δbfe were measured as 12.0±0.6 mM, 9.5±0.7 mM and 11.6±0.6 mM respectively (FIG. 19f). The reduction of flavin by bfe gene knockout in MR-1 Δbfe only produced 31% power and the increase of flavin by overexpression of ribADEHC in MR-1/YYDT-C5 boosted power generation by 2-fold, in comparison with MR-1 WT (Table 2). These results demonstrate that flavin-enabled electron shuttling was the dominant mechanism of bioelectricity generation in MR-1, which is in-agreement with previous study¹⁸. It also suggests that the introduction of the gene cluster encoding flavin biosynthesis (e.g. pYYDT-C5) into an established biofilm of MFCs has the potential to significantly enhance electricity production performance.

Ultrasound-Based DNA Delivery (UDD) in Microbial Fuel Cells (MFC)

The transfer of pYYDT-C5 plasmid into MR-1 WT biofilms via UDD in MFC was investigated employing the setup represented in FIG. 17b. The effect of pYYDT-C5 on bioelectricity generation in established biofilms was investigated in MFC systems. The treatments included ultrasound and the addition of plasmid (WT_P_US), a positive control MR-1/YYDT-C5 strain (MR-1/YYDT-C5_US), and two negative controls: the addition of plasmid (WT_P) without ultrasound and ultrasound treatment without plasmid (WT_US).

As with the previous experiment, the electricity generation of MR-1/YYDT-C5 positive control system (28.0±3.3 μA/cm²) was consistently higher than the MR-1 WT system throughout the experiment (FIG. 20a). After current generation reached steady state after approximately 4 days for all MFC systems, the addition of plasmid and/or ultrasound treatment was conducted between day 5 and 6. Current production in all treated MFC systems dropped immediately after ultrasound treatment was performed, but current production was fully recovered after approximately 24 hrs (FIG. 20). This observation indicates that ultrasound treatment can result in temporary disturbance of the MFC system, possibly mild physical disruption of biofilm structure, but cells in biofilm were able to restructure themselves and fully recover with no permanent detectable detriment afterwards.

Forty-eight hrs after ultrasound treatment, the WT_P_US system started to generate higher current than that of WT_US. At the end of the experiment, the WT_P_US system generated a current of 21.9±1.2 μA/cm², 61% higher (p<0.05) than that of the WT_US system (13.6±1.6 μA/cm²) (FIG. 20a; Table 3). This suggests the successful application of UDD in the established biofilms within the MFC, resulting in the increased production of flavins by the WT_P_US system over time. The WT_P system produced similar current to WT_US (14.9±0.6 μA/cm²), indicating that bacterial transformation only occurred in treatments where both plasmids and ultrasound addition were combined. Three days after the ultrasound treatment, kanamycin (10 μg/mL) and lactate (10 mM) were added into all reactors as selective pressure for transformed cell growth and to maintain high electron donors concentration, respectively. We estimate that a time gap of 3 days was sufficient to enable the transformed bacterial cells, which were maintained at room temperature, to produce the necessary proteins in low-growth minimum media to resist the antibiotics. Additional kanamycin (40 μg/mL) was added on day 13. The addition of antibiotics on the separate occasions had no detectable effects on the current produced by the controls, since the mode of action of kanamycin did not initiate immediate killing of cells but instead interferes with protein synthesis and prevents cell replication¹⁶. This indicated that the established cell density in those reactors had reached optimum concentration before the antibiotics were added and that the process was not catalyst limited. Injection of additional lactate on day 9 also had no detectable effect of improving performance on bioelectricity production, indicating that the reaction was not substrate-limited either.

The quantity of flavins secreted by Shewanella strain played a significant role in influencing the current generation in MFC system¹³. After 14 days of operation, the amount of extracellular flavins in each MFC reactor was quantified. The WT_P_US system generated about 50% higher concentration (p<0.05) of extracellular flavins (103.3±8.3 μM) compared to the WT_US and WT_P systems (70.9±5.9 μM, and 74.8±7.3 μM respectively) (FIG. 4b, Table 3). The enhanced flavin production in WT_P_US system was attributed to the additional synthesis pathway encoding on pYYDT-C5 plasmid, which was introduced into Shewanella oneidensis biofilm via UDD. This quantity analysis of flavin confirmed the transfer of the plasmid via ultrasound. MR-1/YYDT-C5 positive control system contained the greatest concentration of flavin (289.7±57.7 μM), which is consistent with the current generation result.

The extraction and sequencing of plasmids from transformed cells in the WT_P_US MFC system (Table 8) provided additional evidence for the successful transfer of the pYYDT-C5 plasmid into S. oneidensis in MFC biofilm. These results combined provide strong evidence of the ability of UDD to deliver desired genes in situ into bacterial biofilm. This demonstrates that such UDD is able to enhance biofilm-based bioelectrochemical performance in MFCs without the need of re-building biofilm, which is highly desirable for industrial large-scale applications.

Discussion

In-Situ Plasmid Uptake by Bacterial Cell in Flow Cell Biofilms Via UDD

In-situ bacterial transformations in biofilms were usually limited to competent cells²⁰. In this study, we attempted to non-invasively and remotely introduce gene into mature biofilm in-situ using ultrasound mediated gene transfer (UDD).

A pBBR1MCS-2_P_{Lux}_sfGFP plasmid (8822 bp) was constructed using the broad-host-range cloning vector backbone pBBR1MCS-2 and DNA fragments encoding sfGFP and the positive-feedback luxI and luxR system.²¹ sfGFP and the positive-feedback luxI and luxR system was used here as they provide a strong green fluorescence signal in transformed bacteria cells.²² This pBBR1MCS-2_P_{Lux}_sfGFP plasmid was employed as delivery DNA for P. putida UWC1 biofilms grown in commercially available microfluidic flow cells systems (FIG. 17a). UWC1 was used in ultrasound-mediated DNA delivery (UDD) because it is an environmentally and industrially relevant bacterium and is not naturally competent.²⁴ We have shown that applying low frequency ultrasound (40 kHz) to biofilms in the presence of plasmids results in the in-situ uptake of pBBR1MCS-2_P_{Lux}_sfGFP plasmid by bacterial cells, which developed green fluorescence signals within the biofilm after 5 hours of incubation (FIG. 18).

However, the key bottleneck of all transformation techniques, including conventional ones such as electroporation and chemical transformation, is low transformation efficiency where it ranges typically between 10⁻⁹ to 10⁻⁵ transformant per cell¹⁷. With such low ratio of transformant as compared to non-transformed cells within the biofilm, it would not be expected for UDD to have much of an impact on the overall functionality of the biofilm. To overcome this challenge, one option was the use of selection pressure after UDD application to restrict the growth of non-transformed cells and allow transformed cells with the selected fitness to multiply more freely within the biofilm.

In this study, kanamycin was used as a selection pressure to enhance the impact of UDD on the general functionality of the biofilms, as seen from the increasing magnitude of green fluorescence signals over time (FIGS. 18a, b, c and g). This method is particularly useful in applications such as industrial wastewater treatment, where different contaminants that may induce toxic shock in the biofilms within bioreactors. For instance, sudden surges of copper content in wastewater may induce toxic shock in biofilms²⁵ and upset their bioreactors, leading to long bioreactor downtime and the release of untreated wastewater into the environment. Current mitigation methods to protect the environment and prevent government regulatory penalties include dilution to reduce copper concentration per unit wastewater and/or procurement of specially formulated sludge to treat the high copper, both incurring huge financial costs and significant bioreactor downtime. The UDD technique described may potentially bridge the gap by transforming bacteria in the biofilm and sludge bioreactors to express the appropriate functionality (e.g. copper resistance) over time in the presence of a selection pressure (e.g. copper).

We have developed an UDD method for bacterial transformation within established biofilms in microfluidic flow cells. With this method, bacteria cells within established biofilms can acquire specific genes of interest (in this case, luxI, luxR and sfGFP) through bacterial transformation, allowing the biofilms to display new phenotypes and functionalities. Our previous work focus on bacteria transformation for cells in suspension¹⁷, and UDD has not been demonstrated in biofilms prior to this work. It has since been shown by others that the same method is able to transfer nucleic acids into Gram-positive bacteria²⁶, but similarly for cells in suspension. The advantage of ultrasound for gene transfer over conventional methods such as electroporation and chemical transformation is that it is more suitable for scale-up for industrial use.

UDD Induced In-Situ Bacterial Transformation in MFC

Employing S. oneidensis MR-1 strains of varying flavin production and bioelectricity generation capabilities, enabled the establishment of a double-compartment MFC reactor system which allowed reliable evaluation of bioelectricity output by strains. It laid the foundation of employing the MFC system to evaluate the potential impact of UDD on the bioelectricity generation by MR-1. MR-1 was selected as a model organism due to its unique extracellular electron transfer ability²⁷ and the fact that it is not naturally competent. pYYDT-C5 plasmid was used for delivery DNA to S. oneidensis MR-1 WT as the plasmid contains the entire flavin biosynthesis gene cluster ribADEHC cloned from Bacillus subtilis, which was previously shown to improve the bioelectricity generation of the transformed MR-1 as compared to the MR-1 WT¹³.

We have shown that applying low frequency ultrasound (40 kHz) to S. oneidensis biofilms growing on electrodes in the presence of plasmids results in the in-situ uptake of pYYDT-C5 plasmid by bacterial cells, which generated almost twice as much bioelectricity in the MFC after 8 days of incubation as compared to negative controls. The pYYDT-C5 plasmid used here is a relatively large plasmid (10450 Bp). While it is well recognised that transformation efficiency decreases with increasing plasmid size²⁸, our results showed that the UDD technology is not limited to the delivery of small plasmid but is also effective for relatively large plasmids as well. Since UDD seems to be a physical phenomenon where cell membrane permeability is acoustically enhanced¹⁷, it is possible that bacterial transformation via UDD involving the uptake of mega-plasmids can be achieved²⁹.

UDD-treated biofilms in MFC were only able to match around 70% of the level of bioelectricity generated by MR-1/YYDT-C5 positive control system by day 14. Compared to the results for the application of UDD in flow cells biofilm, it is evident that bacterial transformation efficiency can be a limiting factor preventing treated biofilms from achieving the maximum theoretical productivity. To alleviate this limitation, appropriate use of selective pressure can be used to amplify the effects of UDD treatment on the biofilm to exhibit high productivity.

It was previously suggested that the mechanism of transdermal protein delivery using low frequency ultrasound, such as 20 kHz, is attributed mainly to cavitational effects.^{30, 31, 32} In a similar vein, it is possible that the mechanism of UDD in biofilms is via acoustic cavitation where microbubbles, formed on the surface of and/or within biofilms, that can oscillate or implode^{33, 34}, resulting in temporary porosity in the cell membrane. The biofilm matrix, containing extracellular polymeric substances such as lipids, polypeptides and polysaccharides of diverse chemical charges, is an ideal adsorption material for the extracellular DNA or plasmids of interest to be introduced to the biofilms. The high cell density in the biofilms, potentially coupled with proximity between the bacteria and plasmids of interest in the biofilm matrix, provide a suitable environment for acoustic-enhanced horizontal gene transfer to take place within non-competent bacterial biofilm communities.

Scaling Up UDD in Biofilms for Industrial Applications

The goal of this study was to introduce new functionalities in established biofilms in bioreactors of different scales via in-situ UDD. UDD-induced gene transfer on biofilms grown in both microbial flow cells and MFC system was successfully demonstrated, with working volumes of 0.16 cm³ and 300 cm³ respectively, demonstrating a scale-up of 1875 times in operating volume. These results provide good evidence that UDD has enormous promise in terms of bacterial transformation at industrial scale. DNA fragments containing genes of interest may be introduced in-situ into established biofilms cultured in bioreactors, reducing downtime and ensuring continuous operations. UDD can also be deployed in the fields where native biofilm communities established in contaminated soils can be augmented by genes known to be effective at biodegradation or metal resistance. It would also be possible to influence gut microbiome of animals and human beings for agricultural or medical purposes using this approach. Thus, the ability to influence the phenotype of established biofilms creates new possibilities in controlling their behaviour in environmental, industrial and even medical settings.

Material and Methods

Chemicals, Bacteria and Plasmids

All chemicals are from Sigma-Aldrich (United Kingdom) and used without modification unless otherwise stated. The strains and plasmids used in this study are listed in Table 1.

pBBR1MCS-2_P_{Lux}_sfGFP plasmid (8822 bp), containing the broad-host-range cloning vector backbone pBBR1MCS2, sfGFP and the positive-feedback luxI and luxR system, was employed as delivery DNA for P. putida UWC1 while pYYDT-C5 (10450 bp, provided by Yang et al¹³), containing entire flavin biosynthesis gene cluster ribADEHC, was employed as delivery DNA for S. oneidensis MR-1 WT. Briefly, plasmid DNA were extracted and purified from bacterial cultures at their respective mid-exponential phase using a QIAprep Spin Miniprep kit (QIAGEN, Germany). DNA concentration was determined using a NanoQuant Plate™ and Spark microplate reader (TECAN, Switzerland). More information on plasmid preparation can be found in supplementary information (SI).

Construction of pBBR1MCS-2_P_{Lux}_sfGFP Plasmid

pTD103luxI_sfGFP (from Jeff Hasty, Addgene plasmid #48885; http://n2t.net/addgene:48885; RRID:Addgene_48885) was cut via restriction digest using BglII then AvrII. The Pₗᵤₓ_sfGFP fragment, containing sfGFP and the positive-feedback luxI and luxR system, was isolated following separation via gel electrophoresis. Pₗᵤₓ_sfGFP was ligated into a pBBR1MCS-2 plasmid backbone (from Kenneth Peterson, Addgene plasmid #85168; http://n2t.net/addgene:85168; RRID:Addgene_85168) which had been linearised by restriction digest with BamHI and XbaI. The resulting pBBR1MCS-2_Pₗᵤₓ_sfGFP plasmid was transformed into C2987 NEB-5α E. coli which were screened via M13 colony PCR. Plasmids were extracted from positive transformants.

Growth of P. putida UWC1 Biofilms

Biofilms of P. putida UWC1 were grown in three-channel flow cells (channel dimensions, 1×4×40 mm³; Merck, Germany) using 1/10^{th}-strength LB medium (Lennox) continuously supplied through a peristaltic pump. The flow system, consisting of 2L glass bottles (Fisher Scientific, United Kingdom), Masterflex silicone tubing and peristaltic pump (Cole-Palmer, United Kingdom), bubble trap and flowcells (Merck, Germany), was assembled and sterilized as described previously.²³ Each flow cell channel was inoculated with 0.3 mL overnight culture (diluted to an OD₆₀₀ of 0.1) using a 1 mL syringe and 26G needle (BD, U.S.A.). After inoculation, the medium flow was stopped for 1 h to allow initial attachment followed by continuous media flow with a flow rate of 10 mL/h.

Ultrasound DNA Delivery Into Flow Cell Biofilms

A total of four sets of flow cells were used to cultivate biofilms where 3-day-old biofilms were treated under 4 conditions: with both addition of plasmid and ultrasound treatment (+P/+U), with only ultrasound treatment (−P/+U), with only addition of plasmid (+P/−U), and without both addition of plasmid and ultrasound treatment (−P/−U). The peristaltic pump connected to the flow cells were switched off prior to ultrasound treatment. 0.3 mL of 10 mM CaCl₂ solution with or without 1 ug/mL of pBBR1MCS-2_Pₗᵤₓ_sfGFP plasmid (coding for green fluorescence protein) was injected into the appropriate flow cells. Tubing at both ends of the flow cells were clamped and the flow cells were incubated at room temperature for 10 minutes. The flow cells were fully submerged in a 40 kHz Branson 3510 ultrasound water bath (Emerson Electric, U.S.A) and appropriate flow cells were subjected to ultrasound treatment for 10 seconds. After resting for a further 10 minutes, the clamps at both ends of the flow cells were removed and the peristaltic pump was switched back on at a flow rate of 10 mL/h. After 2 hours of flow, the growth media were changed to 1/10th-strength LB medium containing 10 μg/mL kanamycin for the rest of the experiment and waste bottles were replaced as and when required. Biofilms samples within the flow cells were viewed using ZEISS LSM 900 with Airyscan 2 confocal laser-scanning microscope (Carl Zeiss AG, Germany) for signs of green fluorescence signal.

Bacterial samples were collected from within the flowcells using sterile needles and syringes, resuspended in sterile 0.9% NaCl solution, and spread on LB agar plates containing 50 μg/mL kanamycin. Any colonies formed on the agar plates were resuspended in sterile 0.9% NaCl solution and underwent plasmid extraction procedure using Monarch® Plasmid Miniprep Kit (New England Biolabs, United Kingdom) according to manufacturer's instructions. Concentration of plasmid samples were determined using a NanoQuant Plate™ and Spark microplate reader (TECAN, Switzerland), while size of plasmids in samples were compared with pBBR1MCS-2_PLux_sfGFP using horizontal gel electrophoresis systems (Bio-Rad, United Kingdom) according to manufacturer's instructions.

MFC Reactor Setup

Double-compartment MFC reactors with a working volume of 300 mL was used to investigate current production. The anode was made of 3.0×3.0 cm2 carbon cloth (H23, 95 g/m2, Quintech). The cathode was carbon cloth with Pt catalyst (1 mg/cm2, PtC 60%, 2.5×4.0 cm2; FuelCellStore). Titanium wire was used to connect the electrodes to the outside of the reactors. Nafion© 117 was used as the exchange membrane to separate the two compartments.

Reactors were assembled and initially filled with deionised water, then autoclaved to achieve sterility. The water was then replaced with appropriate media; Standard M9 minimal salt, supplemented with trace minerals, amino acids and vitamins was chosen as the anodic compartment media and prepared according to Cao et al.¹⁷ with slight modifications. The list of chemicals and their corresponding concentrations in each stock are given in Tables 4, 5, and 6. The M9 salt solution was autoclaved before the trace elements were added in 1:100 dilution from their stocks via 20 um pore-size membrane sterile filtration. The final medium was supplemented with 20 mM sodium DL-lactate and 0.75 mM IPTG as pYYDT-C5 plasmid inducer. Cathodic compartment media was phosphate buffer saline (PBS), prepared by dissolving two 500 mg PBS tablets in 1 L deionised water, then autoclaved to achieve sterility.

Fixed resistors of 1 kΩ were used to complete the circuit. Keithley Instrument Datalogger 2701 was used to measure the voltage across the resistor every 10 minutes. Before bacterial injection, the anodic compartment was bubbled with nitrogen for 15 minutes to create anaerobic condition. Throughout the experiment, the anodic and cathodic compartments was continuously gassed with nitrogen and air, respectively. Three independent replicate reactors were run for each different system.

Polarisation and Power Curve Construction

The power production of wild-type S. oneidensis MR-1 and its flavin deficient/enhancement mutant counterparts was measured via polarisation curve construction. A potentiostat (PalmSens 4-channel Multi EmStat³⁺) was used to perform Linear Sweep Voltametry (LSV) on the MFC reactors, with the voltage varied between the theoretical open-circuit potential to zero. (E_{begin}=0.8V, E_{end}=0.0V, Eₛₜₑₚ=0.1V, scan rate=0.1 mV/s). Power curve was constructed by multiplying the resulted current with its corresponding potential according to Ohm's law, with the power normalised by the anode surface area

P=IV (1)

Where P is power, I is electric current and V is the applied potential.

Planktonic and Biofilm Cell Quantification

The concentration of planktonic cells in the reactor was determined by its optical density using light spectrometer (UV-1800 Shimadzu), at wavelength of 600 nm. Cuvette length of 1 cm with sample size of 1 mL was used, with fresh anodic media as blank to exclude background reading.

Biofilm cell concentration was measured using crystal violet assay. Anode was immersed in 20 mL of 0.1% crystal violet solution, then washed with 20 mL sterile deionised water twice. Finally, the cell bound crystal violet was dissolved in 20 mL of 70% isopropanol. Four independent replicates of 100 μL aliquot of the final solution was measured for its absorbance at 595 nm, and normalised with background reading of crystal violet originating from a cell-free anode. The OD₅₉₅ value is proportional to the number of cells attached on the biofilm, with the OD-to-cell number conversion was calculated using standard curve of known cell density.

Metabolites Quantification and Coulombic Efficiency

The amount of remaining lactate and produced metabolites were quantified via high-performance liquid chromatography (HPLC) equipped with acid column Hi Plex-H (250×4.6 mm, particle size 8 μm, Agilent). The eluent was 0.005 M H₂SO₄ with flow rate of 0.6 mL/min, and signal was detected using UV detector at 210 nm and 55° C. 1 mL of reactors' medium was sampled and filtered using 0.2 ul pore-size membrane filter to remove cells before being measured for its chemical concentration. Prior to the MFC experiment, standard curves of lactate and possible metabolites (acetate, pyruvate, format and succinate) were constructed.

Coulombic efficiency was calculated as the ratio of charge recovered as electric current to the total theoretical number of charge available from oxidation of lactate to acetate. Recovered electrons as electric current was measured as the integration of current over time

Qᵣ=∫₀^{t}Idt (2)

Where Qᵣ is total charge recovered as current and t is the total duration of operation. And total number of electrons available from lactate oxidation is

Q_{A}=zFVΔC (3)

Where Q_{A} is the total available charge, z is the no of electrons released per molecule of lactate oxidised (z=4), F is the Faraday constant (96,485 C mol⁻¹) V is the anodic compartment volume and ΔC is the change in lactate concentration.

In-Situ Plasmid Transfer Into S. oneidensis MR-1 in MFC

The effect of pYYDT-C5 plasmid transfer into S. oneidensis MR-1 via ultrasound was investigated in terms of the current production of its MFC system. Late-stationary phase culture of MR-1 was injected into the reactor to achieve an initial OD of 0.01. After reaching stable current generation across 1 kΩ resistor, 0.1 μg/mL of the plasmid was injected into appropriate reactors (WT_P_US). Ultrasound was then performed for 30 s at frequency 42 kHz (±6%) to transfer the plasmid into the cell, and current production was continued to be monitored. As controls, reactors with wild-type (WT_US) and MR-1/YYDT-C5 strain (MR-1/YYDT-C5_US) without further addition of plasmid were also experimented as controls. Another control of WT strain with plasmid addition, but without ultrasound treatment, was also measured to exclude the effect of such treatment (WT_P). Three independent replicate reactors were run for each system (target and three controls; 12 reactors in total). Injection of kanamycin and lactate was done using sterile syringe and needle through one of the ports on the side of the reactor. Kanamycin was added from 50 mg/mL stock to achieve desired final concentration in the reactor. Lactate was added from its 1M stock, pre-filter sterilised to achieve sterility.

The efficiency of plasmid transfer was calculated at the end of the experiment. 20 mL of anodic cultures were sampled and centrifuged to obtain cell pellet. The cells were resuspended in 100 μL sterile water then plated on LB agar with 50 μg/mL kanamycin. The numbers of colonies formed were counted and this represented the cells which had obtained the plasmid. The plasmid transfer efficiency was calculated with reference to the total number of cells, transformed and non-transformed, based on its OD value and OD-to-cell number conversion that had been determined previously.

Flavin Quantification

Fluorescence spectroscopy was used to detect and quantify riboflavin and flavin mononucleotide (FMN) secreted by S. oneidensis in the MFC reactor. 100 μL of the cell-free supernatant of anodic media was transferred to a clear 96-well plate and read at 440 nm excitation and 525 nm emission. Four independent replicate aliquots were run for each reactor, and the background fluorescence was corrected by using fresh anodic media as the blank. Flavin concentration was determined using standard curves previously constructed with known concentrations of FMN (concentration range: 1 mg mL⁻¹ to 1 ng mL⁻¹).

Plasmid Sequencing and Verification

At the end of MFC experiment, the anodic biofilm was collected and centrifuged to obtain cell pellets. Plasmid extraction protocol using Monarch® Plasmid Miniprep Kit was performed and the obtained plasmid was quantified using NanoDrop and plate reader. Primers PRTac-SF3_for and ribC-02_R8_rev (Table 7) was used to sequence and identify the necessary plasmid fragment to confirm successful transfer of pYYDT-C5 plasmid into S. oneidensis.

Statistical Analysis

For all measurements involving replication, nested mixed-factor ANOVA test followed by Tukey's HSD post hoc test was performed to study the significance between the different treatment groups. P value of less than 0.05 denotes statistically significant difference between the systems of interest.

### REFERENCES

1. Stoodley P, Sauer K, Davies D G, Costerton J W. Biofilms as complex differentiated communities. Annual Reviews in Microbiology 56, 187-209 (2002).

2. Meckenstock R U, et al. Biodegradation: updating the concepts of control for microbial cleanup in contaminated aquifers. (ed\hat{ }(eds). ACS Publications (2015).

3. Halan B, Buehler K, Schmid A. Biofilms as living catalysts in continuous chemical syntheses. Trends Biotechnol 30, 453-465 (2012).

4. Botyanszki Z, Tay P K R, Nguyen P Q, Nussbaumer M G, Joshi N S. Engineered catalytic biofilms: Site-specific enzyme immobilization onto E. coli curli nanofibers. Biotechnology and Bioengineering, n/a-n/a (2015).

5. Singh R, Paul D, Jain R K. Biofilms: implications in bioremediation. Trends Microbiol 14, 389-397 (2006).

6. Stewart P S, Franklin M J. Physiological heterogeneity in biofilms. Nat Rev Microbiol 6, 199-210 (2008).

7. Elias S, Banin E. Multi-species biofilms: living with friendly neighbors. FEMS Microbiology Reviews 36, 990-1004 (2012).

8. Hays S G, Patrick W G, Ziesack M, Oxman N, Silver P A. Better together: engineering and application of microbial symbioses. Current opinion in biotechnology 36, 40-49 (2015).

9. Rosche B, Li X Z, Hauer B, Schmid A, Buehler K. Microbial biofilms: a concept for industrial catalysis? Trends Biotechnol 27, 636-643 (2009).

10. Pagga U, Bachner J, Strotmann U. Inhibition of nitrification in laboratory tests and model wastewater treatment plants. Chemosphere 65, 1-8 (2006).

11. Sorensen S J, Bailey M, Hansen L H, Kroer N, Wuertz S. Studying plasmid horizontal transfer in situ: a critical review. Nature Reviews Microbiology 3, 700-710 (2005).

12. Brim H, et al. Engineering Deinococcus radiodurans for metal remediation in radioactive mixed waste environments. Nature biotechnology 18, 85-90 (2000).

13. Yang Y, et al. Enhancing bidirectional electron transfer of Shewanella oneidensis by a synthetic flavin pathway. ACS synthetic biology 4, 815-823 (2015).

14. Collard J-M, et al. Plasmids for heavy metal resistance in Alcaligenes eutrophus CH34: mechanisms and applications. FEMS microbiology reviews 14, 405-414 (1994).

15. Leonard S P, et al. Genetic engineering of bee gut microbiome bacteria with a toolkit for modular assembly of broad-host-range plasmids. ACS synthetic biology 7, 1279-1290 (2018).

16. De Lorenzo V. Recombinant bacteria for environmental release: what went wrong and what we have learnt from it. Clinical microbiology and infection 15, 63-65 (2009).

17. Song Y, et al. Ultrasound-mediated DNA transfer for bacteria. Nucleic Acids Research 35, e129-e129 (2007).

18. Kotloski N J, Gralnick J A. Flavin electron shuttles dominate extracellular electron transfer by Shewanella oneidensis. MBio 4, e00553-00512 (2013).

19. Liu T, et al. Enhanced Shewanella biofilm promotes bioelectricity generation. Biotechnol Bioeng 112, 2051-2059 (2015).

20. Hendrickx L, Hausner M, Wuertz S. Natural genetic transformation in monoculture Acinetobacter sp. strain BD413 biofilms. Applied and environmental microbiology 69, 1721-1727 (2003).

21. Prindle A, Samayoa P, Razinkov I, Danino T, Tsimring L S, Hasty J. A sensing array of radically coupled genetic ‘biopixels’. Nature 481, 39-44 (2012).

22. Scott S R, Din M O, Bittihn P, Xiong L, Tsimring L S, Hasty J. A stabilized microbial ecosystem of self-limiting bacteria using synthetic quorum-regulated lysis. Nature microbiology 2, 1-9 (2017).

23. Sternberg C, Tolker-Nielsen T. Growing and analyzing biofilms in flow cells. Curr Protoc Microbiol, 1B. 2.1-1B. 2.15 (2006).

24. McCLURE N C, Weightman A J, Fry J C. Survival of Pseudomonas putida UWC1 containing cloned catabolic genes in a model activated-sludge unit. Applied and Environmental microbiology 55, 2627-2634 (1989).

25. Cabrero A, Fernandez S, Mirada F, Garcia J. Effects of copper and zinc on the activated sludge bacteria growth kinetics. Water research 32, 1355-1362 (1998).

26. Lin L, et al. Ultrasound-mediated DNA transformation in thermophilic Gram-positive anaerobes. PLoS One 5, (2010).

27. Shi L, Squier T C, Zachara J M, Fredrickson J K. Respiration of metal (hydr)oxides by Shewanella and Geobacter: a key role for multihaem c-type cytochromes. Mol Microbiol 65, 12-20 (2007).

28. Hanahan D. Studies on transformation of Escherichia coli with plasmids. Journal of molecular biology 166, 557-580 (1983).

29. Taghavi S, Van der Lelie D, Mergeay M. Electroporation of Alcaligenes eutrophus with (mega) plasmids and genomic DNA fragments. Applied and environmental microbiology 60, 3585-3591 (1994).

30. Mitragotri S, Blankschtein D, Langer R. Ultrasound-mediated transdermal protein delivery. Science 269, 850-853 (1995).

31. Prausnitz M R, Mitragotri S, Langer R. Current status and future potential of transdermal drug delivery. Nature reviews Drug discovery 3, 115-124 (2004).

32. Tang H, Blankschtein D, Langer R. Effects of low-frequency ultrasound on the transdermal permeation of mannitol: Comparative studies with in vivo and in vitro skin. Journal of pharmaceutical sciences 91, 1776-1794 (2002).

33. Vyas N, et al. Which parameters affect biofilm removal with acoustic cavitation? A review. Ultrasound in medicine & biology 45, 1044-1055 (2019).

34. Erriu M, et al. Microbial biofilm modulation by ultrasound: current concepts and controversies. Ultrasonics sonochemistry 21, 15-22 (2014).

40. McCLURE N C, Weightman A J, Fry J C. Survival of Pseudomonas putida UWC1 containing cloned catabolic genes in a model activated-sludge unit. Appl Environ Microbiol 55, 2627-2634 (1989).

41. Heidelberg J F, et al. Genome sequence of the dissimilatory metal ion-reducing bacterium Shewanella oneidensis. Nature biotechnology 20, 1118-1123 (2002).

42. Dehio C, Meyer M. Maintenance of broad-host-range incompatibility group P and group Q plasmids and transposition of Tn5 in Bartonella henselae following conjugal plasmid transfer from Escherichia coli. Journal of bacteriology 179, 538-540 (1997).

43. Kostylev M, Otwell A E, Richardson R E, Suzuki Y. Cloning should be simple: Escherichia coli DH5α-mediated assembly of multiple DNA fragments with short end homologies. PLoS One 10, (2015).

44. Kovach M E, et al. Four new derivatives of the broad-host-range cloning vector pBBR1MCS, carrying different antibiotic-resistance cassettes. Gene 166, 175-176 (1995).

All references herein are incorporated by reference.

Tables

TABLE 1
Bacterial strains and plasmids used in this study.

| | | |
|---|---|---|
| Bacterial strain or | | Reference |
| plasmid | Genotype, description | or source |

Strains

| | | |
|---|---|---|
| Pseudomonas | A spontaneous rifampicin-resistant mutant of P. | 40 |
| putida UWC1 | putida KT2440. Not naturally competent. | |
| UWC1/sfGFP | P. putida UWC1: pBBR1MCS-2_Pₗᵤₓ_sfGFP | |
| Shewanella | Wild type strain of MR-1. Not naturally competent. | 41 |
| oneidensis MR-1 | | |
| wild type (WT) | | |
| MR-1 Δbfe | Δbfe mutant of MR-1. Loss of ability to transport | 18 |
| | the FAD into the periplasm, reduced extracellular | |
| | flavins available for electron transfer. | |
| MR-1/YYDT-C5 | S. oneidensis MR-1: pYYDT-C5 | This study |
| Escherichia coli | A diaminopimelate (DAP) auxotroph due to | 42 |
| WM3064 | mutation in dapA. Cannot undergo cell division | |
| | without DAP. | |
| Escherichia coli | A derivative of the E. coli DH5α. Competent cell | 43 |
| C2987 NEB-5α | for laboratory genetic manipulation, from New | |
| | England Biolabs (U.K.) | |

Plasmid

| | | |
|---|---|---|
| pBBR1MCS-2 | Empty vector backbone with broad host-range | 44 |
| | origin of replication (pBBR1) multiple cloning site | |
| | with blue/white selection function, Kan^{R} | |
| pBBR1MCS- | Plasmid with positive-feedback luxI and luxR | This |
| 2_Pₗᵤₓ_sfGFP | system and superfolding green fluorescence protein | study |
| | (sfGFP), Kan^{R} | |
| pTD1031uxl_sfGFP | Oscillator plasmids with positive-feedback luxI and | 21 |
| | luxR system and superfolding green fluorescence | |
| | protein (sfGFP), colE1, Kan^{R} | |
| pYYDT-C5 | Plasmid with entire flavin biosynthesis gene cluster | 13 |
| | ribADEHC cloned from Bacillus subtilis, Kan^{R} | |

TABLE 2
Steady-state current density and maximum power output
of MFC running with MR-1 wild-type and mutants

| | Current Density | Max. Power Output |
|---|---|---|
| | [μA/cm²] | [μW/cm²] |
| MR-1 WT | 13.7 ± 0.3 | 2.61 ± 0.35 |
| MR-1 Δbfe | 7.6 ± 0.1 | 0.83 ± 0.19 |
| MR-1/YYDT-C5 | 31.5 ± 1.8 | 5.25 ± 1.18 |

TABLE 3
Final current density and extracellular flavin
concentrations of UDD-treated MFC systems

| | Current density | Flavins concentrations |
|---|---|---|
| | [μA/cm²] | [μM] |
| WT_P_US | 21.9 ± 1.2 | 103.3 ± 8.3 |
| WT_US (−ve control) | 13.6 ± 1.6 | 70.9 ± 5.9 |
| WT_P (−ve control) | 14.9 ± 0.6 | 74.8 ± 7.3 |
| MR-1/YYDT-C5_US | 28.0 ± 3.3 | 289.7 ± 57.7 |
| (+ve control) | | |

TABLE 4
Ingredients of vitamin stock (×100)

| Chemical | FW | mg/L |
|---|---|---|
| Biotin (d-biotin) | 244.3 | 2 |
| Folic acid | 441.1 | 2 |
| Pyridoxine HCl | 205.6 | 10 |
| Riboflavin | 376.4 | 5 |
| Thiamine HCl 1.0 H₂O | 355.3 | 5 |
| Nicotinic acid | 123.1 | 5 |
| d-Pantothenic acid, hemicalcium | 238.3 | 5 |
| salt | | |
| B12 | 1355.4 | 0.1 |
| p-Aminobenzoic acid | 137.13 | 5 |
| Thioctic acid (or lipoic acid) | 206.3 | 5 |

TABLE 5
Ingredients of mineral stock (×100)

| Chemical | FW | g/L |
|---|---|---|
| Nitrilotriacetic acid | 199.1 | 1.5 |
| MgSO4•7H2O | 246.48 | 3 |
| MnSO4•H2O | 169.02 | 0.5 |
| NaCl | 58.44 | 1 |
| FeSO4•7H2O | 277.91 | 0.1 |
| CaCl2•2H2O | 146.99 | 0.1 |
| CoCl2•6H2O | 237.93 | 0.1 |
| ZnCl2 | 136.28 | 0.13 |
| CuSO4•5H2O | 249.68 | 0.01 |
| AlK(SO4)2•12H2O | 474.38 | 0.01 |
| H3BO3 | 61.83 | 0.01 |
| Na2MoO4•2H2O | 241.95 | 0.025 |
| NiCl2•6H2O | 237.6 | 0.024 |
| Na2WO4•2H2O | 329.86 | 0.025 |

TABLE 6
Ingredients of amino acid stock (×100)

| Chemical | FW | g/L |
|---|---|---|
| L-Glutamic acid | 147.13 | 2 |
| L-arginine | 174.2 | 2 |
| DL-serine | 105.09 | 2 |

TABLE 7
pYYDT-C5 fragment to be detected by
PRTac-SF3 for and ribC-02_R8_rev
for UDD confirmation in MFC

Forward (SEQ ID NO: 1):
NNNNGGNNNNNNNNAGAGGAGAATCTAGTATGTTC
CACCCAATCGAAGAAGCTTTAGATGCTTTAAAAAA
AGGTGAAGTTATCATCGTTGTTGATGATGAAGATC
GTGAAAACGAAGGTGATTTCGTTGCTTTAGCTGAA
CACGCTACTCCAGAAGTTATCAACTTCATGGCTAC
TCACGGTCGTGGTTTAATCTGTACTCCATTATCTG
AAGAAATCGCTGATCGTTTAGATTTACACCCAATG
GTTGAACACAACACTGATTCTCACCACACTGCTTT
CACTGTTTCTATCGATCACCGTGAAACTAAAACTG
GTATCTCTGCTCAAGAACGTTCTTTCACTGTTCAA
GCTTTATTAGATTCTAAATCTGTTCCATCTGATTT
CCAACGTCCAGGTCACATCTTCCCATTAATCGCTA
AAAAAGGTGGTGTTTTAAAACGTGCTGGTCACACT
GAAGCTGCTGTTGATTTAGCTGAAGCTTGTGGTTC
TCCAGGTGCTGGTGTTATCTGTGAAATCATGAACG
AAGATGGTACTATGGCTCGTGTTCCAGAATTAATC
GAAATCGCTAAAAAACACCAATTAAAAATGATCAC
TATCAAAGATTTAATCCAATACCGTTACAACTTAA
CTACTTTAGTTGAACGTGAAGTTGATATCACTTTA
CCAACTGATTTCGGTACTTTCAAAGTTTACGGTTA
CACTAACGAAGTTGATGGTAAAGAACACGTTGCTT
TCGTTATGGGTGATGTTCCATTCGGTGAANAACCA
GTTTTAGTTCGTGTTCNNTCTGAATGTTTAACTGG
TGATGTTTTCGGTTCTCANCGTTGTGATTGTGGTC
CACAATTACNCGCTGCTTTAAACCAAATCGCTGCT
GAAGGTCGNGGNGTTTNNTAAACTTACGTCANNNA
GGTCNNNGTATCGGTTTAATCANNAAATTAAAAGC
TTANAAATTANNNNAACAAGGTTANAANNNNGNTN
NNNCTANNNNNNNNTNNNNNNNNNNNNNNNNNNNN
NNNNNNNNNNNNNNNNNNNNNNNNNNNNNNNNNNN
NNNNNNNNNANNNNNNNNNNNNNNNNNNNNNCNNN
NANNNNNNNNNNTANNNNNNNNNNNNNNNNNNNNN
NNNNNNNNNNNNNNNNNNNNNNNNNNNAA
Reverse (SEQ ID NO: 2):
NNNNNGCTTCNNNGTNNNCCNTTTTCNNNTTGTTC
AGTTAATTCTTTTGATACCGTTGAATTTACGTTCA
GAACGGATACGTTTGTACCATTCGATTTTGATAGC
AGCACCGTAAACTTCTTTGGTTGAAATCGAATAAG
TTAACTTCGATAGATGGTTGTTTCTGGACGTTTTT
CGTAGAAAGTTGGTTTGTAACCGATGTTACAAACA
CCGTTTGTAAACTTCACCGTTAACTTCAGCTTTAA
CAGCGTAAACACCAGTTGGTGGAACGATGTAAGAG
TTGTTTAAACCAACGTTAGCAGTTGGGAAACCGAT
AGTACGACCACGTTTATCACCGTGGATAACGATAC
CTTTGATGAAGTATGGTTGACCTAATAAAACGTTA
GCTAATTCAACATCACCGTTTTGTAAAGCAGTACG
GATGTAAGAAGAAGAGATTTTTTTATCTTGTTCAG
TTAATTTTTCAACCATAGTACAACCAGCTTTACCA
TCTAAATCATCTGGCATAGTTTTCATAGTACCTTT
ACCGTATTTACCGTAAGTGAAATCGAAACCAGCAA
CAGCGTGTTGAACGTTTAAACCGATGATGTATTGA
TCGATGAATTGTTTTGGAGATAAAGAAGCGAAAAC
TTCGTTGAATTTAACAACGTATAAAACTTCAGTAC
CTAATTGTTCGATTTGGTTGATTTTATCTTCTAAT
GGAGTGATTAAATCTTTTGGTTCTTTATCACGACC
TAAAACGTGAGATGGGTGTGGGTGGAAAGTCATAA
CAGCTAAAGTTAAACCTTTTTCTTCAGCGATTTGT
TTAGCAGTACCGATAACTTTTTGGTGACCTAAGTG
AANNCCATCGAAGTAACCTAAAGCCNNAACAGATT
TAGCTTGNTCTCNTTGATAATGGNGGGGNNNNNNN
NNNGGGGAAANTNTNNNCNNNAGTTNNNNCNNNNN
NNNNNNNNTANNNNAAANAACGGTTANNTNNNNNN
TNNNNNNNNNNNNNNTTGACTANNNNACANATNNN
NNNN

TABLE 8
Sequencing result of UDD-treated
MFC-biofilm plasmid

Forward (SEQ ID NO: 3):
NNNNGGGNNNNGAAGAGGAGAATCTAGTATGTTCC
ACCCAATCGAAGAAGCTTTAGATGCTTTAAAAAAA
GGTGAAGTTATCATCGTTGTTGATGATGAAGATCG
TGAAAACGAAGGTGATTTCGTTGCTTTAGCTGAAC
ACGCTACTCCAGAAGTTATCAACTTCATGGCTACT
CACGGTCGTGGTTTAATCTGTACTCCATTATCTGA
AGAAATCGCTGATCGTTTAGATTTACACCCAATGG
TTGAACACAACACTGATTCTCACCACACTGCTTTC
ACTGTTTCTATCGATCACCGTGAAACTAAAACTGG
TATCTCTGCTCAAGAACGTTCTTTCACTGTTCAAG
CTTTATTAGATTCTAAATCTGTTCCATCTGATTTC
CAACGTCCAGGTCACATCTTCCCATTAATCGCTAA
AAAAGGTGGTGTTTTAAAACGTGCTGGTCACACTG
AAGCTGCTGTTGATTTAGCTGAAGCTTGTGGTTCT
CCAGGTGCTGGTGTTATCTGTGAAATCATGAACGA
AGATGGTACTATGGCTCGTGTTCCAGAATTAATCG
AAATCGCTAAAAAACACCAATTAAAAATGATCACT
ATCAAAGATTTAATCCAATACCGTTACAACTTAAC
TACTTTAGTTGAACGTGAAGTTGATATCACTTTAC
CAACTGATTTCGGTACTTTCAAAGTTTACGGTTAC
ACTAACGAAGTTGATGGTAAAGAACACGTTGCTTT
CGTTATGGGTGATGTTCCATTCGGTGAANAACCAG
TTTTAGTTCGNGTTCACTCTGAATGTTTAACTGNN
GATGTTTTCGGTTCTNACCGTTGTGATTGTGGTCC
ACAATTACNCGNTGCTTTAAACCAAATCGCTGCTG
AAGGTCGNNNTNTTTTATNANACTTACGTCANNNA
GGTNNNGGTNTCGGTTNAATCAACAAATTAAAAGC
TTACAATTACANNNACAAGGTTANAAANNNNNNNN
NNNTAANNANNNNNNNNNNNNNNNNNNNNNNNNNN
NNNNNNNNNNNNNNNNNNNNNNNNNNNNNNNNNNN
NNNNNNNNNNNNNNNANNNNNNNCNNNNNNNNNNN
NNNANNNNNNNNNNNNNNNNNNNNNNNNNNNNNNN
NNNNNA
Reverse (SEQ ID NO: 4):
NNNNNCTTCTTTGTTTATCTTTTTCGATTTGTTCA
GTTAATTCTTTGATACCGTTGAATTTACGTTCAGA
ACGGATACGTTTGTACCATTCGATTTTGATAGCAG
CACCGTAAACTTCTTGGTTGAAATCGAATAAGTTA
ACTTCGATAGATGGTTGTTCTGGACGTTTTTCGTA
GAAAGTTGGTTTGTAACCGATGTTACAAACACCGT
TGTAAACTTCACCGTTAACTTCAGCTTTAACAGCG
TAAACACCAGTTGGTGGAACGATGTAAGAGTTGTT
TAAACCAACGTTAGCAGTTGGGAAACCGATAGTAC
GACCACGTTTATCACCGTGGATAACGATACCTTTG
ATGAAGTATGGTTGACCTAATAAAACGTTAGCTAA
TTCAACATCACCGTTTTGTAAAGCAGTACGGATGT
AAGAAGAAGAGATTTTTTTATCTTGTTCAGTTAAT
TTTTCAACCATAGTACAACCAGCTTTACCATCTAA
ATCATCTGGCATAGTTTTCATAGTACCTTTACCGT
ATTTACCGTAAGTGAAATCGAAACCAGCAACAGCG
TGTTGAACGTTTAAACCGATGATGTATTGATCGAT
GAATTGTTTTGGAGATAAAGAAGCGAAAACTTCGT
TGAATTTAACAACGTATAAAACTTCAGTACCTAAT
TGTTCGATTTGGTTGATTTTATCTTCTAATGGAGT
GATTAAATCTTTTGGTTCTTTATCACGACCTAAAA
CGTGAGATGGGGTGTGGGGTGGAAAGTCATAACAG
CTAAAGTTAAACCTTTTTCTTCAGCGATTTGTTTA
GCAGTACCGATAACTTTTTGGTGACCTAAGTGAAC
ACCATCGAAGTAACCTAAAGCNATAACNNNTTTAG
NTTGNTCTTCTTTGATTAAKNGNGGGGGTGAATGA
NNNNNAAAANTTT

1. A method for the transformation of host cells of a biofilm with heterologous nucleic acid, wherein the host cells are within the extracellular matrix of the biofilm, the method comprising:
adding the heterologous nucleic acid to the biofilm; and
applying inertial cavitation to the biofilm in the presence of the heterologous nucleic acid to facilitate transformation of host cells within the biofilm with the heterologous nucleic acid.

2. The method according to claim 1, wherein the level of inertial cavitation activity is monitored by sensing the acoustic cavitation noise and that information is used to adjust exposure parameters in real time.

3. The method according to claim 1 or claim 2, wherein the biofilm is in situ.

4. The method according to claim 1 or claim 2 or claim 3, wherein an enclosure is applied to the biofilm when adding the heterologous nucleic acid to the biofilm.

5. The method according to claim 4, wherein the enclosure comprises an access port for administration/delivery of the heterologous nucleic acid, and/or other substances, into the enclosure.

6. The method according to claim 4 or claim 5, wherein the enclosure comprises a secondary enclosure that is arranged to retain and release the heterologous nucleic acid and/or other substances into the enclosure.

7. The method according to any preceding claim, further comprising an incubation period of at least 30 seconds between adding the heterologous nucleic acid and applying the ultrasound.

8. The method according to any preceding claim, wherein the biofilm is located in a watercourse, a channel, a pipe, a pellicle, an oil or water feed, a stream, a river, a water body, a reactor, a dispersed/suspended growth system, an attached growth system, an aquifer, the internal and/or external body of a ship or boat, soil crumbs, a plant leaf surface or plant roots; or wherein the biofilm is in a microbial fuel cell (MFC).

9. The method according to any preceding claim, wherein the inertial cavitation is induced by application of ultrasound.

10. The method according to any preceding claim, wherein the heterologous nucleic acid is a plasmid or vector.

11. The method according to any preceding claim, wherein the heterologous nucleic acid encodes a gene and/or or a regulatory element that is capable of modifying the host cell phenotype.

12. The method according to any preceding claim, wherein the heterologous nucleic acid encodes a gene and/or regulatory element of a gene involved in, or that is arranged to modify one or more functions from the group comprising, quorum sensing, cell metabolism, heat/cold resistance, heat-shock resistance, chemical resistance, antibiotic resistance, cell aggregation, cell adhesion, cell export, membrane transport molecules, cell or EPS dispersal enzymes, and stress regulons; or a combination thereof; or wherein the heterologous nucleic acid encodes a redox pathway or one or more parts thereof.

13. The method according to any preceding claim, wherein the heterologous nucleic acid encodes an enzyme, a membrane transporter, a pore molecule, and/or a regulatory element associated therewith.

14. The method according to any preceding claim, wherein the heterologous nucleic acid encodes a gene selected from any of the group comprising protein-degrading enzymes, such as protease and peptidase; polysaccharide-degrading enzymes and oligosaccharide-degrading enzymes, such as endocellulase, chitinase, α-glucosidase, β-glucosidase, β-xylosidase, N-acetyl-β-d-glucosaminidase, chitobiosidase, and β-glucuronidase; lipid-degrading enzymes, such as lipase and esterase; phosphomonoesterases, such as phosphatase; oxidoreductases, such as phenol oxidase, peroxidase; and extracellular redox activity; or combinations thereof; or wherein the heterologous nucleic acid encodes a one or more, or all, genes of the gene cluster mtrCAB or ribADEHC.

15. The method according to any preceding claim, wherein the heterologous nucleic acid is arranged to promote survival or growth of a selected species of bacteria in the biofilm relative to other species.

16. The method according to any preceding claim, wherein the heterologous nucleic acid is applied to the biofilm in a CaCl₂ solution, or in the presence of CaCl₂.

17. A method of adapting a biofilm in situ, the method comprising transformation of host cells within the extracellular matrix of the biofilm with heterologous nucleic acid,
wherein the method comprises:
adding the heterologous nucleic acid to the biofilm; and
applying inertial cavitation to the biofilm in the presence of the heterologous nucleic acid to facilitate transformation of host cells within the biofilm with the heterologous nucleic acid,
wherein the heterologous nucleic acid encodes a gene and/or or a regulatory element that is capable of modifying the host cell phenotype, or the heterologous nucleic acid is arranged to knockout a host cell gene, or regulatory sequence thereof, of the host cell.

18. The method according to claim 17, wherein an enclosure is applied to the biofilm, and the heterologous nucleic acid is added within the enclosure.

19. A method of decontaminating feedstock in a waste water treatment process, the method comprising:
flowing the feedstock over a biofilm
wherein cells of the biofilm have been genetically modified in situ in order to increase their ability to reduce the contaminant, such as an aromatic, in the feedstock and/or increase the resistance of the biofilm to the contaminant in the feedstock.

20. Use of ultrasound to transform host cells within the extracellular matrix of a biofilm.

21. The use according to claim 20, wherein the use is to transform cells of a biofilm in situ.

22. Use of cavitation to transform host cells within the extracellular matrix of a biofilm.

23. The use according to claim 22, wherein the cavitation can be produced from ultrasound or other physical methods.

24. A kit for transformation of host cells within the extracellular matrix of a biofilm, wherein the kit comprises:
an inertial cavitation generator;
an enclosure; and
optionally, nucleic acid for transformation.

25. The kit according to claim 24, further comprising CaCl₂.

26. A method of generating electricity from a microbial fuel cell (MFC) comprising:
culturing bacteria in a biofilm in an anode compartment of the MFC, wherein the bacteria of the biofilm have been transformed with heterologous nucleic acid encoding one or more genes of a redox pathway;
supplying an oxidant and a substrate for oxidation that are substrates of the redox pathway;
generating electricity by allowing electrons released by the bacteria from the substrate oxidation in the anode compartment to be transferred to a cathode compartment of the MFC through a conductive material, whereby the transferred electrons in the cathode compartment are combined with oxygen and the protons are diffused through a proton exchange membrane.


SEQUENCE LISTING (4 sequences)

SEQ ID NO: 1 - DNA, 1149 bp, Shewanella oneidensis
  misc_feature (1)..(4): n is a, c, g, or t
  misc_feature (7)..(14): n is a, c, g, or t
  misc_feature (765)..(765): n is a, c, g, or t
  misc_feature (787)..(788): n is a, c, g, or t
  misc_feature (824)..(824): n is a, c, g, or t
  misc_feature (850)..(850): n is a, c, g, or t
  misc_feature (884)..(884): n is a, c, g, or t
  misc_feature (887)..(887): n is a, c, g, or t
  misc_feature (892)..(893): n is a, c, g, or t
  misc_feature (907)..(909): n is a, c, g, or t
  misc_feature (915)..(917): n is a, c, g, or t
  misc_feature (933)..(934): n is a, c, g, or t
  misc_feature (949)..(949): n is a, c, g, or t
  misc_feature (956)..(959): n is a, c, g, or t
  misc_feature (970)..(970): n is a, c, g, or t
  misc_feature (973)..(976): n is a, c, g, or t
  misc_feature (978)..(978): n is a, c, g, or t
  misc_feature (980)..(983): n is a, c, g, or t
  misc_feature (987)..(994): n is a, c, g, or t
  misc_feature (996)..(1059): n is a, c, g, or t
  misc_feature (1061)..(1081): n is a, c, g, or t
  misc_feature (1083)..(1086): n is a, c, g, or t
  misc_feature (1088)..(1097): n is a, c, g, or t
  misc_feature (1100)..(1147): n is a, c, g, or t
nnnnggnnnnnnnnagaggagaatctagtatgttccacccaatcgaagaagctttagatgctttaaaaaaaggtgaagttatcatcgttgttgatgatgaagatcgtgaaaacgaaggtgatttcgttgctttagctgaacacgctactccagaagttatcaacttcatggctactcacggtcgtggtttaatctgtactccattatctgaagaaatcgctgatcgtttagatttacacccaatggttgaacacaacactgattctcaccacactgctttcactgtttctatcgatcaccgtgaaactaaaactggtatctctgctcaagaacgttctttcactgttcaagctttattagattctaaatctgttccatctgatttccaacgtccaggtcacatcttcccattaatcgctaaaaaaggtggtgttttaaaacgtgctggtcacactgaagctgctgttgatttagctgaagcttgtggttctccaggtgctggtgttatctgtgaaatcatgaacgaagatggtactatggctcgtgttccagaattaatcgaaatcgctaaaaaacaccaattaaaaatgatcactatcaaagatttaatccaataccgttacaacttaactactttagttgaacgtgaagttgatatcactttaccaactgatttcggtactttcaaagtttacggttacactaacgaagttgatggtaaagaacacgttgctttcgttatgggtgatgttccattcggtgaanaaccagttttagttcgtgttcnntctgaatgtttaactggtgatgttttcggttctcancgttgtgattgtggtccacaattacncgctgctttaaaccaaatcgctgctgaaggtcgnggngtttnntaaacttacgtcannnaggtcnnngtatcggtttaatcannaaattaaaagcttanaaattannnnaacaaggttanaannnngntnnnnctannnnnnnntnnnnnnnnnnnnnnnnnnnnnnnnnnnnnnnnnnnnnnnnnnnnnnnnnnnnnnnnnnnnnnnnannnnnnnnnnnnnnnnnnnnncnnnnannnnnnnnnntannnnnnnnnnnnnnnnnnnnnnnnnnnnnnnnnnnnnnnnnnnnnnnnaa

SEQ ID NO: 2 - DNA, 1019 bp, Shewanella oneidensis
  misc_feature (1)..(5): n is a, c, g, or t
  misc_feature (11)..(13): n is a, c, g, or t
  misc_feature (16)..(18): n is a, c, g, or t
  misc_feature (21)..(21): n is a, c, g, or t
  misc_feature (27)..(29): n is a, c, g, or t
  misc_feature (843)..(844): n is a, c, g, or t
  misc_feature (866)..(867): n is a, c, g, or t
  misc_feature (883)..(883): n is a, c, g, or t
  misc_feature (888)..(888): n is a, c, g, or t
  misc_feature (899)..(899): n is a, c, g, or t
  misc_feature (904)..(913): n is a, c, g, or t
  misc_feature (921)..(921): n is a, c, g, or t
  misc_feature (923)..(923): n is a, c, g, or t
  misc_feature (925)..(927): n is a, c, g, or t
  misc_feature (929)..(931): n is a, c, g, or t
  misc_feature (936)..(939): n is a, c, g, or t
  misc_feature (941)..(953): n is a, c, g, or t
  misc_feature (956)..(959): n is a, c, g, or t
  misc_feature (963)..(963): n is a, c, g, or t
  misc_feature (972)..(973): n is a, c, g, or t
  misc_feature (975)..(980): n is a, c, g, or t
  misc_feature (982)..(995): n is a, c, g, or t
  misc_feature (1003)..(1006): n is a, c, g, or t
  misc_feature (1010)..(1010): n is a, c, g, or t
  misc_feature (1013)..(1019): n is a, c, g, or t
nnnnngcttcnnngtnnnccnttttcnnnttgttcagttaattcttttgataccgttgaatttacgttcagaacggatacgtttgtaccattcgattttgatagcagcaccgtaaacttctttggttgaaatcgaataagttaacttcgatagatggttgtttctggacgtttttcgtagaaagttggtttgtaaccgatgttacaaacaccgtttgtaaacttcaccgttaacttcagctttaacagcgtaaacaccagttggtggaacgatgtaagagttgtttaaaccaacgttagcagttgggaaaccgatagtacgaccacgtttatcaccgtggataacgatacctttgatgaagtatggttgacctaataaaacgttagctaattcaacatcaccgttttgtaaagcagtacggatgtaagaagaagagatttttttatcttgttcagttaatttttcaaccatagtacaaccagctttaccatctaaatcatctggcatagttttcatagtacctttaccgtatttaccgtaagtgaaatcgaaaccagcaacagcgtgttgaacgtttaaaccgatgatgtattgatcgatgaattgttttggagataaagaagcgaaaacttcgttgaatttaacaacgtataaaacttcagtacctaattgttcgatttggttgattttatcttctaatggagtgattaaatcttttggttctttatcacgacctaaaacgtgagatgggtgtgggtggaaagtcataacagctaaagttaaacctttttcttcagcgatttgtttagcagtaccgataactttttggtgacctaagtgaannccatcgaagtaacctaaagccnnaacagatttagcttgntctcnttgataatggnggggnnnnnnnnnnggggaaantntnnncnnnagttnnnncnnnnnnnnnnnnntannnnaaanaacggttanntnnnnnntnnnnnnnnnnnnnnttgactannnnacanatnnnnnnn

SEQ ID NO: 3 - DNA, 1126 bp, Shewanella oneidensis
  misc_feature (1)..(4): n is a, c, g, or t
  misc_feature (8)..(11): n is a, c, g, or t
  misc_feature (764)..(764): n is a, c, g, or t
  misc_feature (781)..(781): n is a, c, g, or t
  misc_feature (804)..(805): n is a, c, g, or t
  misc_feature (821)..(821): n is a, c, g, or t
  misc_feature (849)..(849): n is a, c, g, or t
  misc_feature (852)..(852): n is a, c, g, or t
  misc_feature (883)..(885): n is a, c, g, or t
  misc_feature (887)..(887): n is a, c, g, or t
  misc_feature (894)..(894): n is a, c, g, or t
  misc_feature (896)..(896): n is a, c, g, or t
  misc_feature (907)..(909): n is a, c, g, or t
  misc_feature (914)..(916): n is a, c, g, or t
  misc_feature (920)..(920): n is a, c, g, or t
  misc_feature (927)..(927): n is a, c, g, or t
  misc_feature (957)..(959): n is a, c, g, or t
  misc_feature (969)..(969): n is a, c, g, or t
  misc_feature (973)..(983): n is a, c, g, or t
  misc_feature (987)..(988): n is a, c, g, or t
  misc_feature (990)..(1065): n is a, c, g, or t
  misc_feature (1067)..(1073): n is a, c, g, or t
  misc_feature (1075)..(1088): n is a, c, g, or t
  misc_feature (1090)..(1125): n is a, c, g, or t
nnnngggnnnngaagaggagaatctagtatgttccacccaatcgaagaagctttagatgctttaaaaaaaggtgaagttatcatcgttgttgatgatgaagatcgtgaaaacgaaggtgatttcgttgctttagctgaacacgctactccagaagttatcaacttcatggctactcacggtcgtggtttaatctgtactccattatctgaagaaatcgctgatcgtttagatttacacccaatggttgaacacaacactgattctcaccacactgctttcactgtttctatcgatcaccgtgaaactaaaactggtatctctgctcaagaacgttctttcactgttcaagctttattagattctaaatctgttccatctgatttccaacgtccaggtcacatcttcccattaatcgctaaaaaaggtggtgttttaaaacgtgctggtcacactgaagctgctgttgatttagctgaagcttgtggttctccaggtgctggtgttatctgtgaaatcatgaacgaagatggtactatggctcgtgttccagaattaatcgaaatcgctaaaaaacaccaattaaaaatgatcactatcaaagatttaatccaataccgttacaacttaactactttagttgaacgtgaagttgatatcactttaccaactgatttcggtactttcaaagtttacggttacactaacgaagttgatggtaaagaacacgttgctttcgttatgggtgatgttccattcggtgaanaaccagttttagttcgngttcactctgaatgtttaactgnngatgttttcggttctnaccgttgtgattgtggtccacaattacncgntgctttaaaccaaatcgctgctgaaggtcgnnntnttttatnanacttacgtcannnaggtnnnggtntcggttnaatcaacaaattaaaagcttacaattacannnacaaggttanaaannnnnnnnnnntaannannnnnnnnnnnnnnnnnnnnnnnnnnnnnnnnnnnnnnnnnnnnnnnnnnnnnnnnnnnnnnnnnnnnnnnnnnnnannnnnnncnnnnnnnnnnnnnnannnnnnnnnnnnnnnnnnnnnnnnnnnnnnnnnnnna

SEQ ID NO: 4 - DNA, 923 bp, Shewanella oneidensis
  misc_feature (1)..(5): n is a, c, g, or t
  misc_feature (862)..(862): n is a, c, g, or t
  misc_feature (868)..(870): n is a, c, g, or t
  misc_feature (876)..(876): n is a, c, g, or t
  misc_feature (880)..(880): n is a, c, g, or t
  misc_feature (895)..(896): n is a, c, g, or t
  misc_feature (898)..(898): n is a, c, g, or t
  misc_feature (911)..(915): n is a, c, g, or t
  misc_feature (920)..(920): n is a, c, g, or t
nnnnncttctttgtttatctttttcgatttgttcagttaattctttgataccgttgaatttacgttcagaacggatacgtttgtaccattcgattttgatagcagcaccgtaaacttcttggttgaaatcgaataagttaacttcgatagatggttgttctggacgtttttcgtagaaagttggtttgtaaccgatgttacaaacaccgttgtaaacttcaccgttaacttcagctttaacagcgtaaacaccagttggtggaacgatgtaagagttgtttaaaccaacgttagcagttgggaaaccgatagtacgaccacgtttatcaccgtggataacgatacctttgatgaagtatggttgacctaataaaacgttagctaattcaacatcaccgttttgtaaagcagtacggatgtaagaagaagagatttttttatcttgttcagttaatttttcaaccatagtacaaccagctttaccatctaaatcatctggcatagttttcatagtacctttaccgtatttaccgtaagtgaaatcgaaaccagcaacagcgtgttgaacgtttaaaccgatgatgtattgatcgatgaattgttttggagataaagaagcgaaaacttcgttgaatttaacaacgtataaaacttcagtacctaattgttcgatttggttgattttatcttctaatggagtgattaaatcttttggttctttatcacgacctaaaacgtgagatggggtgtggggtggaaagtcataacagctaaagttaaacctttttcttcagcgatttgtttagcagtaccgataactttttggtgacctaagtgaacaccatcgaagtaacctaaagcnataacnnntttagnttgntcttctttgattaanngngggggtgaatgannnnnaaaanttt